(19) United States
(12) Patent Application Publication
(10) Pub. No.: US 2026/0106024 A1
(43) Pub. Date: Apr. 16, 2026

(54) SYSTEM AND METHOD FOR PATIENT AND HEALTHCARE PROFESSIONAL MATCHING, AND DIAGNOSIS OR TREATMENT PLAN MATCHING OR GENERATION WITH MULTI-LEVEL VERIFICATION USING BLOCKCHAIN

(71) Applicant: James Wang, Alpharetta, GA (US)

(72) Inventor: James Wang, Alpharetta, GA (US)

(21) Appl. No.: 18/913,212

(22) Filed: Oct. 11, 2024

(51) Int. Cl. G16H 50/20; G06F 21/62; G06Q 10/0639; G16H 15/00; G16H 20/10; G16H 30/40; G16H 40/67

(52) U.S. Cl. CPC G16H 50/20; G06F 21/6263; G06Q 10/06398; G16H 15/00; G16H 20/10; G16H 30/40; G16H 40/67; G06Q 2220/00

(57) ABSTRACT

A method for automated patient and healthcare professional and services matching, and automated matching or generation of a diagnosis, treatment plan and medication recommendation may be provided. On top of the AI empowered network for this healthcare application, a smart contract operating on a blockchain-based distributed computer network is proposed which may obtain public and private information for a patient or healthcare professional. The smart contract may provide the public information via a public access layer in response to a public request. The smart contract may provide the private information via a privileged access layer in response to a verified request, wherein the patient may control data access to the privileged layer. The smart contract may provide public or private information via the public or privileged layer in response to a verified request regarding a treatment, medication, or medical condition.

### FIELD OF DISCLOSURE

The following relates generally to automated healthcare services, and more specifically to automated matching of patients to healthcare providers, to diagnoses, to treatment plans to provide healthcare services. In some embodiments, such services may be provided using transactions on a blockchain-based network.

### BACKGROUND

Conventional healthcare systems may face challenges in efficiently connecting patients with appropriate medical providers. The process of finding a suitable physician for a specific medical condition may often be time-consuming and may not always result in an optimal match. This may lead to delays in treatment and potentially suboptimal care outcomes.

Additionally, traditional methods of storing and sharing medical records may present privacy and security concerns. Patient data may be vulnerable to unauthorized access or breaches when stored in centralized databases. Furthermore, patients may have limited control over who can access their medical information and how it is used.

The diagnosis and treatment recommendation process in conventional healthcare systems may also be subject to inefficiencies. Physicians may need to manually review patient histories and symptoms, which may be time-consuming and may not always leverage the full potential of available medical knowledge and data.

Moreover, healthcare transactions and processes may involve multiple intermediaries and manual steps. This may result in increased administrative costs and potential delays in care delivery. The lack of a streamlined, automated system for managing these processes may contribute to overall inefficiencies in the healthcare system.

Accordingly, there is a need for an intelligent way to match patients with appropriate healthcare providers, as well as with appropriate diagnosis and treatment plans when the patient information is provided. There also exists a need for a streamlined and secure way to track patient medical records, generate diagnoses and treatment plans, and implement those plans, with the delivery of the treatment promptly.

AI technology is applying rigorous machine learning models and various AI algorithms to calculate, simulate, optimize and try resolve real world issues. Yet, there is limited real AI usage in sophisticated healthcare applications, especially the above indicated real situations and needs. Blockchain technology is a decentralized, distributed ledger that records the transactions and ownership of a digital asset. A blockchain is essentially a chain of blocks, each containing data, and that are linked together using cryptography. Blockchains enable a secure record of data and are designed to generate trust in transactions without the need for a trusted third party. This technology underpins various cryptocurrencies and can be used for a wide range of applications beyond currencies including supply chain management, digital identity verification, voting systems, and transferring ownership rights of other assets.

Some available blockchain technologies extend their functionality with the use of smart contracts. Smart contracts are self-executing contracts with the terms of the agreement between buyer and seller being directly written into lines of code. Some smart contracts operate without a buyer or seller, e.g., in response to information from an oracle or in response to a query. They operate on blockchain technology and are automatically executed, controlled, and documented by the blockchain when pre-defined conditions are met. Smart contracts eliminate the need for intermediaries, thus reducing transaction costs and increasing transaction speed and transparency.

Accordingly, there is a need for an AI-based system that is capable of streamlining the process of matching patients with healthcare providers that are well-versed in the needs of the patient. There is also a need for an AI-based system that is useful for patients and/or their healthcare providers to propose diagnoses and/or treatment plans based on patient medical data, helping to streamline the healthcare process and allowing patients to efficiently receive care needed for any current injury or illness.

### SUMMARY

This brief overview is provided to introduce a selection of applications in a simplified form that are further described below in the Detailed Description. This brief overview is not intended to identify key features or essential features of the claimed subject matter. Nor is this brief overview intended to be used to limit the claimed subject matter's scope.

The present disclosure describes methods for automatically matching patients to appropriate healthcare professionals, such as (but not limited to) physicians, physical therapists, psychologists, nurses, and/or other healthcare providers, as well as to appropriate diagnoses, treatment plans, and the like. This matching can be achieved through a uniquely designed machine learning and AI configuration. In some embodiments, the method may include matching to or generating one or more diagnoses, providing one or more treatment plans, and/or responding to queries related to healthcare needs according to a privilege hierarchy. Embodiments are further configured to automatically update existing diagnoses and/or treatment plans, to execute transactions involving the patient and a healthcare professional, and to match patients to appropriate healthcare professional, or to diagnoses and/or treatment plans in a secure manner where patient privacy is maintained. Embodiments include an AI empowered/enhanced computer network that may aid in running a healthcare system. The process may be automatically run based on the AI matches generated (e.g., certain diagnoses can easily be derived or matched from information provided by patients; and the treatment plan for the diagnosis may also be easily determined). Embodiments can also include a smart contract component configured to deploy various smart contracts into a blockchain-based distributed computer network, where the smart contracts include code from or otherwise incorporate one or more machine learning (ML) models. The smart contracts are further able to add blocks to the blockchain with varying levels of access, effectively creating a privileged access layer of the blockchain.

In embodiments, a method, apparatus, non-transitory computer readable medium, and/or system for automated or semi-automated provision of healthcare services may include (but need not be limited to) matching patients with healthcare providers, with/or generating one or more patient diagnoses, and/or establishing one or more patient treatment plans on a blockchain-based network, or just on AI processing network without a blockchain-based network. One or more aspects of the method, apparatus, non-transitory computer readable medium, and system include obtaining, by a smart contract operating on a blockchain-based distributed computer network, information regarding a patient information, which may include general demographic information (e.g., name, address, age, height, weight, race, gender, etc.), condition information (e.g., medications taken by the patient, current patient diagnoses, patient test results, etc.). A first subset of the patient information may be public information, and a second subset of the patient information may be private information. In embodiments, the method may include providing, by the smart contract, the public information via a public access layer of the blockchain-based distributed computer network for processing in response to a public request for information about the patient; and providing, by the smart contract, the private information via a privileged access layer of the blockchain-based distributed computer network for processing in response to a verified request for information about the patient.

In other embodiments, an apparatus, system, and/or method for automated management of healthcare diagnoses, treatments, and/or medications on a blockchain-based network are described. One or more aspects of the apparatus, system, and method include at least one processor; at least one memory storing instructions executable by the at least one processor; a public blockchain component configured to provide and/or process public information via a public access layer of a blockchain-based distributed computer network in response to a public request for information about a healthcare process where the request does not include private information (e.g., a notice of food poisoning that does not identify the patient); and a private of privileged blockchain component configured to provide and process private information via a privileged access layer of the blockchain-based distributed computer network in response to a verified request for information about an illness, diagnosis, treatment plan, and/or medication including (but not limited to) treatment of chronic illness, physical injury, infectious disease, cancer, etc., processing test results and/or other medical data. The privileged access layer may transmit and/or process private information including data unavailable to the public (via the public access layer) For example, the privileged access layer may be capable of transmitting and/or processing patient personal information (PII), medical history, symptoms, etc. The privileged access layer may be capable of running private processes for the patient including (but not limited to) generating treatment plans and/or advice for patient or a healthcare professional treating the patient, collecting information on improvement of symptom after treatment, generating a proposed treatment plan including one or more proposed medications, any related doctor input and advice, information of full recovery afterwards, etc. In this private layer, a patient's private healthcare data is stored, and the patient has control to give access to relatives, healthcare professionals, medical institutions, insurance providers etc.

A method for automated patient and healthcare professional matching, and automated matching or generation of a treatment plan and medication recommendation, may be provided. The method may obtain, by a smart contract operating on a blockchain-based distributed computer network, public information for a patient or healthcare professional, and private information for the patient or healthcare professional. The method may provide, by the smart contract, the public information via a public access layer of the blockchain-based distributed computer network in response to a public request for information about the patient or the healthcare professional. The method may provide, by the smart contract, the private information via a privileged access layer of the blockchain-based distributed computer network in response to a verified request for information about the patient or healthcare professional, wherein the patient may control data access to the privileged layer. The method may provide, by the smart contract, one or more of the public information or the private information via one or more of the public access layer or the privileged access layer of the blockchain-based distributed computer network in response to a verified request for information regarding one or more of: a treatment, a medication regarding any symptom, or a medical condition.

A non-transitory computer readable medium storing code may be provided. The code may comprise instructions executable by a processor to perform operations. The operations may obtain, by a smart contract operating on a blockchain-based distributed computer network, public information for a healthcare service and private information for the healthcare service. The operations may provide, by the smart contract, the public information via a public access layer of the blockchain-based distributed computer network in response to a public request for information about the healthcare service. The operations may provide, by the smart contract, the private information via a privileged access layer of the blockchain-based distributed computer network in response to a verified request for information about the healthcare service.

A non-transitory computer-readable medium storing instructions may be provided. When executed by one or more processors, the instructions may cause the one or more processors to perform operations. The operations may receive patient data comprising medical history, symptoms, and demographic information. The operations may analyze the patient data using an artificial intelligence (AI) model trained on a dataset of historical patient records and medical outcomes. The operations may generate, based on the analysis, a ranked list of potential diagnoses for the patient. The operations may identify, using the AI model, a set of suitable healthcare professionals for the patient based on the potential diagnoses and healthcare professional specialties. The operations may match the patient with a recommended healthcare professional from the set of suitable healthcare professionals based on one or more of availability, patient location, and historical success rates for treating similar conditions. The operations may generate a personalized treatment plan for the patient using the AI model, wherein the treatment plan may be based on the recommended healthcare professional's expertise and the patient's specific medical profile. The operations may transmit the potential diagnoses, recommended healthcare professional and personalized treatment plan to a user device associated with the patient. The operations may update the AI model based on feedback received regarding the accuracy of the diagnoses, effectiveness of the doctor match, and success of the treatment plan.

Both the foregoing brief overview and the following detailed description provide examples and are explanatory only. Accordingly, the foregoing brief overview and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicant. In addition, the drawings may contain other marks owned by third parties and are being used for illustrative purposes only. All rights to various trademarks and copyrights represented herein, except those belonging to their respective owners, are vested in and the property of the Applicant. The Applicant retains and reserves all rights in its trademarks and copyrights included herein, and grants permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure. In the drawings:

FIG. 1 shows an example of a healthcare service matching system according to aspects of the present disclosure.

FIG. 2 shows an example of a healthcare service matching apparatus according to aspects of the present disclosure.

FIG. 3 shows an example of a pipeline for smart contract creation according to aspects of the present disclosure.

FIG. 4 shows an example of a pipeline for listing generation according to aspects of the present disclosure.

FIG. 5 shows an example of a pipeline for patient and healthcare professional matching according to aspects of the present disclosure.

FIG. 6 shows an example of a pipeline for treatment generation according to aspects of the present disclosure.

FIG. 7 shows an example of a pipeline for medication generation according to aspects of the present disclosure.

FIG. 8 shows an example of public and private layers according to aspects of the present disclosure.

FIG. 9 shows an example of a pipeline for diagnosis matching or generation models according to aspects of the present disclosure.

FIG. 10 shows an example of a method for retrieving privileged information according to aspects of the present disclosure.

FIG. 11 shows an example of a method for automated healthcare provider, treatment, and/or medication matching and generation according to aspects of the present disclosure.

FIG. 12 shows an example of a computing device according to aspects of the present disclosure.

### DETAILED DESCRIPTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure and are made merely to provide a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection is to be defined by the issued claim(s) rather than the description set forth herein.

Additionally, it is important to note that each term used herein refers to that which an ordinary artisan would understand such a term to mean based on the contextual use of the term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Regarding applicability of 35 U.S.C. § 112, ¶ 6, no claim element is intended to be read in accordance with this statutory provision unless the explicit phrase “means for” or “step for” is actually used in such claim element, whereupon this statutory provision is intended to apply in the interpretation of such claim element.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subject matter disclosed under the header.

The present disclosure includes many aspects and features. Moreover, while many aspects and features relate to, and are described in, the context of a healthcare service matching platform, embodiments of the present disclosure are not limited to use only in this context.

Blockchain technology provides a secure, immutable ledger for recording transactions and ownership of digital assets. Some of the available blockchains further provide smart contract compatibility, enabling the automation of transactions and data generation. However, conventional blockchain systems do not differentiate users based on their access-level privilege. Accordingly, the conventional systems are not suited for transactions necessitating private or confidential data. For example, healthcare providers may require sensitive information about patients such as medical records and/or other personal information. Moreover, healthcare providers in the United States owe a special duty to maintain private information according to Health Insurance Portability and Accountability Act (HIPPA) regulations. Other locations may have similar regulations. For example, a patient may have provided or requested sensitive information about their illness, treatment, medication, etc. that cannot be disclosed to the general public, or that the patient wishes to keep secret from the general public. In such cases, the data should be stored securely, such that the patient can control authorization for accessing the data. For example, the patient may allow access to designated relatives, healthcare professionals, medical institutions, insurance providers, and/or the like.

The present disclosure includes methods for enabling multi-level verification on a blockchain-based distributed computer network. The methods provided herein may be applied to many domains. However, the present disclosure focuses on methods for automating and facilitating healthcare services, controlling private data access, matching patients to healthcare providers, generating suggested diagnoses and/or treatment plans for users (patients and/or healthcare providers), and enabling various healthcare services for users (patients and/or healthcare providers).

For example, if a patient is suffering from a physical injury (e.g., a sports injury, such as a muscle strain or ligament sprain), the processes of the present disclosure include management of a diagnosis matching and generation of a treatment plan using a blockchain-based distributed computer network, or simply on a system with automated matching but without blockchain-based network. The process may include a method for a patient to provide private information (e.g., including patient image, video and other information regarding symptom, cause of injury, etc.), utilizing machine learning models and/or artificial intelligence (AI) methods to generate a suggested diagnosis and/or a suggested treatment plan for the patient based at least in part on the provided information, and providing the generated information and support to the patient and/or a designated healthcare professional in a secure and efficient manner through the blockchain system.

In another example, if a patient is suffering from symptoms of an infectious disease (e.g., cold, respiratory system infection, etc.) the processes of the present disclosure may include managing diagnosis matching and treatment process on a blockchain-based distributed computer network, or simply on a system with automated matching but without blockchain-based network.. The processes may include receiving public and/or private information from the patient, which may include (but need not be limited to) patient image and/or video data, patient information regarding symptoms, possible causes of infection, and/or the like. One or more machine learning models and/or AI methods may be used to generate a suggested diagnosis (e.g., cold, Covid-19, and/or other complex respiratory disease), and/or a suggested treatment plan. The process may use AI to match the patient with a healthcare provider to confirm the suggested diagnosis and treatment and may provide the information to the patient and/or the matched healthcare professional (with patient approval) in a secure and efficient manner through this blockchain system.

In another example, if a patient is suffering from chronic disease (e.g., various cancers and/or other chronic disease), the processes of the present disclosure include managing the diagnosis matching and treatment processes using a blockchain-based distributed computer network, or simply on a system with automated matching but without blockchain-based network. The processes may include receiving, from the patient, private information which may include (but need not be limited to) patient image and/or video data, and/or other information regarding symptoms, history changes in condition, and/or the like. The processes may utilize machine learning models and AI methods to generate suggested diagnoses or suggest updates thereto, to suggest new treatment plans or modifications to a current treatment plan, and/or to match a patient with a healthcare provider. The healthcare provider may review and confirm or adjust the diagnoses and/or treatment plans. The process may provide the generated information to the patient and/or a designated healthcare provider in a secure and efficient manner through this blockchain system.

AI technologies with uniquely sophisticated artificial intelligence models is developed here to generate optimized matches and healthcare processing. For example, here a multimodal transformer-based architecture is designed that integrates patient history and various data modalities to enable effective patient-doctor matching, diagnosis, and treatment recommendation. This enables the efficient allocation of medical resources, accurate diagnosis, and personalized treatment recommendations.

This architecture consists of a multimodal transformer model that can process and fuse information from various data modalities, such as structured electronic health records (EHRs), unstructured clinical notes, medical images, and patient-reported outcomes (PROs). This transformer model, such as BERT (Bidirectional Encoder Representations from Transformers) or its variants, is pre-trained on a large corpus of medical literature and fine-tuned on task-specific datasets.

A conventional process treating patient maladies (injuries, illnesses, etc.) involves the patient first engaging with a healthcare professional, such as a physician. Currently, the patient has little control over the process, leaving their data in the hands of the healthcare professional. Most patients are not aware of where their private data is stored or how to access the data. Moreover, because the patient must select a healthcare provider prior to being diagnosed, they may not have access to all information needed to select a healthcare provider that is particularly suited to their malady. For example, while any internist may be competent to treat a common cold or pneumonia, it may be desirable to see a pulmonologist if patient symptoms are more reminiscent of COPD. Moreover, healthcare professionals typically lack the capacity to locate patients that specifically need their specialized services. Rather, patients and healthcare providers rely on a complex network of referrals. Further, medical records are currently stored and shared in ways that do not promote efficiency. This can reduce overall efficiency and take time away from both patients and healthcare professionals.

When a patient first visits a healthcare professional, the healthcare professional typically collects patient data, such as a medical history, and examines the patient to determine symptoms and arrive at a diagnosis. The healthcare provider may then manually prepare a treatment plan which may include medications, therapies, treatments, surgeries, and/or other ways to alleviate symptoms and/or treat the underlying cause of the symptoms. After following the treatment plan, the patient may return for subsequent visits, where the healthcare provider may check for improvements in the condition and modify the treatment plan accordingly. In cases where the provider is not familiar with the illness affecting the patient, treatment may be slower than optimal. It may take significant time to find an effective treatment to which the patient responds well. In some cases, it may even require a referral to a different healthcare provider to start the entire process over with a more knowledgeable provider.

Thus, in the current healthcare system, the treatment and medication process could be a long and tedious manual process, including multiple provider visits to multiple providers, multiple examinations, and making adjustments to the treatment plan over time. This current process is not only time and resource consuming but may result in the patient seeing a provider who is a poor choice for their condition, incorrect diagnosis of a patient ailment, and/or incorrect treatment for the patient ailment. These sub-optimal matchings cause a waste of resources and can cost patients their health and possibly even their lives. Moreover, the patient's private data and medical history information is not stored and accessed efficiently, resulting in a waste of resources in data storage and/or increased danger of private data leaks exposing the patient's private information, while the patient does not have control of their own information or who can access it.

Blockchain-based distributed computer networks provide an alternative to traditional databases such as patient databases including patient private info (e.g., previous medical history, illness & treatment, known allergies, other healthcare data including data of various treatment and medication, healthcare service vendors and healthcare professional information, preferred pharmacies, etc.). Blockchain systems are more powerful than centralized databases in many aspects. For example, the blockchain systems indicated in this disclosure secure private data superbly while enabling separate access to public &private data, efficiently not compromising either layer. Further, a patient who owns a blockchain wallet containing the patient data can control their own data, including who can access the data.

Blockchain systems can also be scaled indefinitely allowing for essentially unlimited growth. The blockchain system can be used to provide access to users all around the world without needing to transfer the data manually, risking interception or corruption. Existing entries (blocks) to the ledger of a blockchain system cannot be modified by bad actors due to the cryptographic linkage between blocks—if a single block is changed, all subsequent blocks would need to change to be valid, which is practically impossible due to the proof-of-work and proof-of-stake validation systems. Further, blockchains may be extended with smart contracts to add further capabilities, such as automating the suggestion of treatment plans by only enabling treatment plans (e.g., procedure and/or or medications) that meet certain criteria laid out in the smart contract. However, existing systems do not include features for handling private or sensitive information.

Embodiments described herein include one or more privileged layers on the blockchain that allow for the private and secure processing of confidential data while maintaining the transparency and integrity of public data. The private layers are designed to store and handle sensitive information integral to users/patients' personal medical information, such as personal medical history, previous illness & treatment, drug allergic information, previous physicians & medical institutions, family information, insurance information etc. These private layers ensure that confidential data remains protected and accessible only to authorized parties, and patients have full control of their own data. In some cases, the privileged layers may be used to store privileged data that is not necessarily sensitive, but that can be gated to other privileged users. As used herein, “private” is used interchangeably with “privileged.” In some examples, “private” refers to a type of layer that is a subset of a “privileged” type.

For example, with a blockchain-based computer network and data processing being performed on both public and private layers of the blockchain, a healthcare professional can much more efficiently obtain patient medical history and data, once the healthcare professional is verified on the private layer of blockchain, where the patient's confidential medical data can be accessed, once the patient permits the healthcare professional to access the corresponding data. Moreover, a healthcare professional can easily communicate with other healthcare professionals or related medical institutions (e.g., other expert physicians in the field, the patient's previous physician, and/or healthcare provider/medical institutions where the patient has previously received relevant treatment) so as to obtain any necessary previous medical data. The blockchain environment further facilitates a healthcare professional quickly receiving suggested diagnoses and/or treatment plans from an AI system, thus tremendously expediting the treatment process. This allows the healthcare professional to provide more value and support to a patient quickly using the blockchain-based computer network, such that the private and personal treatment needs are addressed smoothly on the private layer of blockchain, while the general information and processing is run on the public layer of blockchain.

In another example, using the blockchain-based computer network and the data processing on both the public and private layers, a patient can receive matches provided by an AI system. The matches may include a listing of a healthcare provider (e.g., a physician/doctor) who is better positioned (e.g., with expertise) to handle the patient's illness (e.g., in terms of diagnosis, treatment and/or medication). In embodiments, a patient can provide illness data (e.g., including image, video, and/or other related information on illness), and easily receive AI system provided diagnosis, treatment and/or medication suggestions, which the patient can then use to consult one or more healthcare professionals for confirmation and preparing treatment. In some embodiments, the patient can receive AI system provided advice on preventative exercises, therapy, and/or other home remedies to reduce or lessen minor physical injury. In some embodiments, the patient can receive AI system provided advice on preventative therapy, treatment, and/or nutrition to reduce or lessen minor illness such as minor cold, allergy, cough, etc. A patient can receive AI system provided prognosis information or projection based on a symptom or illness, enabling the patient to pre-emptively take precautions or take steps to prevent, reduce, or alleviate an illness, symptom, or other malady that may present in the patient. In some situations, the patient can receive AI system provided matching and related advice on a rehabilitation center or facility, to provide therapy or other treatment for an illness or injury. The patient can receive AI system provided matching related to a medical institution, healthcare insurance provider, pharmacy, or other healthcare provider and/or service. Thus, the process of receiving treatment and recovery suggestions for patients can be expedited and streamlined by using one or more of the blockchain-based computer network and the AI system processing. The private, personal information and treatment needs are addressed smoothly on the private layer of blockchain, and general information and process is run on the public layer of blockchain, thus both public and private needs are processed smoothly to address patient needs through this configuration of blockchain-based computer network.

Embodiments improve on treatment & medication process by combining the AI automation or optimization of the treatment, and authentication/verification features of blockchain systems with the privacy features of multi-tiered access layers. This allows patient to have control of their own data or information, healthcare needs & process, and control who has access, who provide treatment etc. And this allows treatment to be generated by smart contracts with access to the privileged data, without exposing the privileged data to the public. Additionally, the integration of AI-driven algorithms within this system enhances the treatment decision-making with efficiency, tailoring treatment and medications to the specific needs of patient. The smart contracts and the trained ML models are powered by having access to the entirety of the blockchain. Alternatively, some embodiments improve on the treatment & medication process using AI automation and/or optimization of the treatment, without blockchain systems.

This structure not only ensures the security and confidentiality of sensitive data but also maintains the transparency and trustworthiness inherent in public blockchain systems. The dual-layered approach of private and public layers in this blockchain architecture streamlines the handling of complex personal information with healthcare and medical related data, which typically involve a mix of public and private information. By segregating this data appropriately, the system ensures that each diagnosis, treatment and medication process adheres to the necessary privacy standards while still benefiting from the immutable and decentralized nature of blockchain technology. This results in a more efficient, secure, and user-centric healthcare process, where user have control of his/her own data, as well as control of who can access what portion of his/her information and addressing key limitations of current blockchain applications in sensitive domains such as healthcare and patient care.

Embodiments include systems and methods for patients to be matched to an appropriate healthcare professional when needed, or healthcare professionals matched to patients who have needs that fall into healthcare professionals' coverage, as well as system to provide some automatic diagnosis process when needed, and further to provide matching diagnosis, treatment and medication with a process that is streamlined with AI-powered smart contracts, or using one or more AI systems without blockchain. Embodiments implement a private or privileged layer onto a blockchain that enables the selective viewing and processing of the information. Some embodiments further ensure that the diagnosis, treatments and medications are compliant with all regulations by generating the content using trained AI models whose outputs are controlled to be compliant. Some embodiments match patients to healthcare professionals, medical institutions, treatment and medications, insurance providers, pharmacies, or to other service providers using trained AI models. Some embodiments further provide analysis, projections, and recommendations based on the information of user or through the medical process.

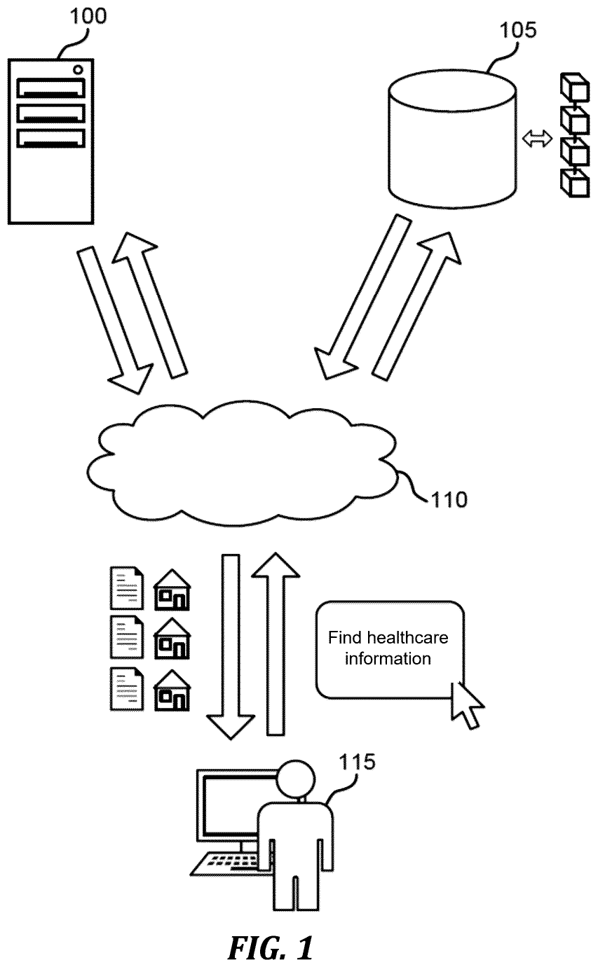

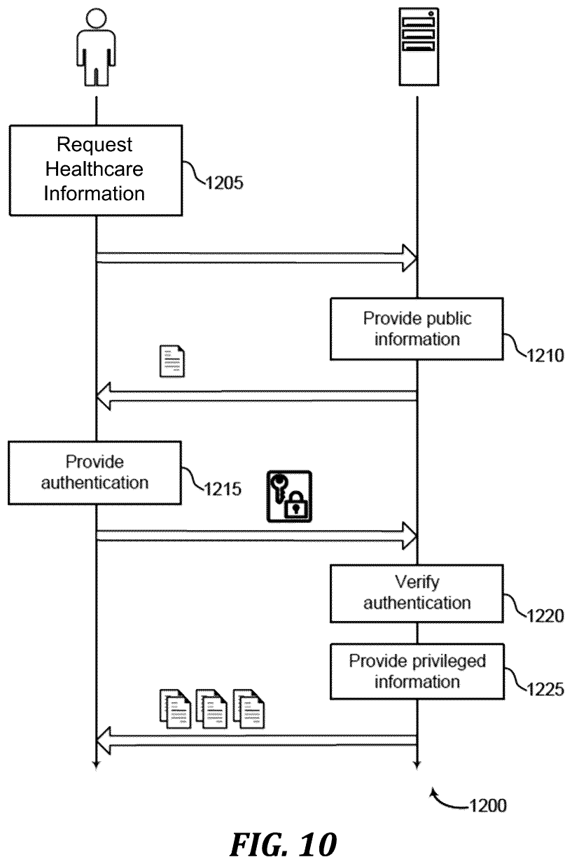

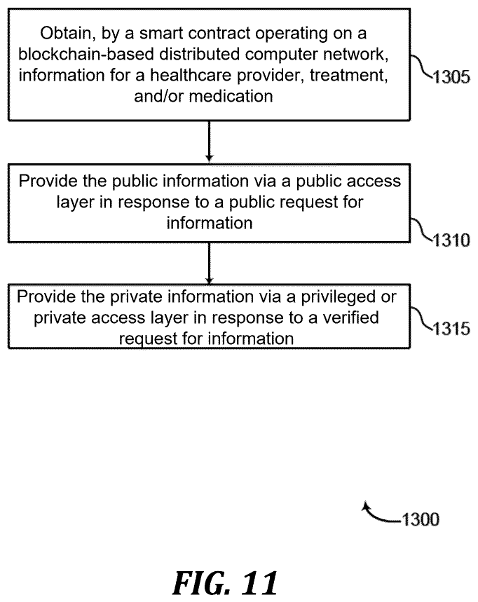

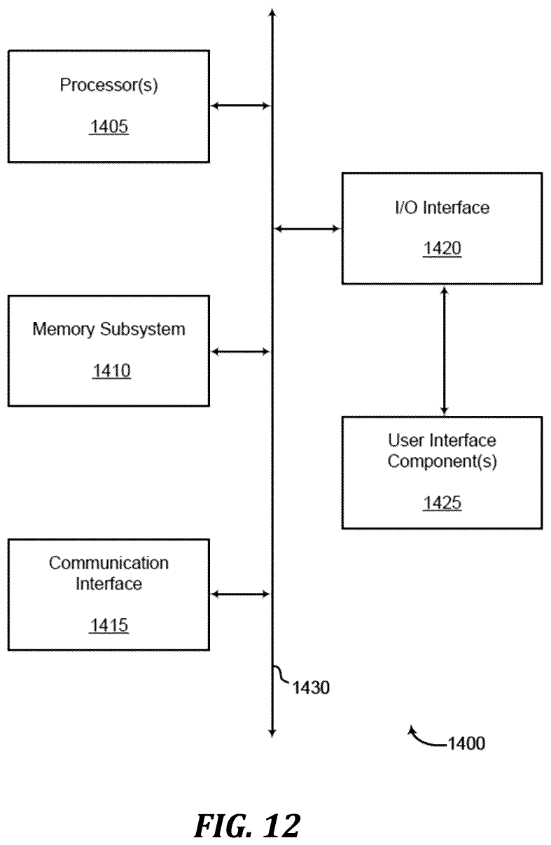

A healthcare service matching system is described with reference to FIGS. 1-9. Matching methods and information query methods are described with reference to FIGS. 10-11. A computing device configured to implement a user/patient matching apparatus is described with reference to FIG. 12.

### Healthcare Service Matching System

The present disclosure may provide a set of modules for facilitating a software and/or hardware platform for patient and healthcare professional matching, diagnosis and treatment plan matching or generation with the option of multi-level verification using blockchain. Details with regard to each module are provided below. Although modules are disclosed with specific functionality, it should be understood that functionality may be shared between modules, with some functions split between modules, while other functions duplicated by the modules. Furthermore, the name of each module should not be construed as limiting upon the functionality of the module. Moreover, each component disclosed within each module can be considered independently, without the context of the other components within the same module or different modules. Each component may contain functionality defined in other portions of this specification. Each component disclosed for one module may be mixed with the functionality of other modules. In the present disclosure, each component can be claimed on its own and/or interchangeably with other components of other modules.

An apparatus for automated healthcare service matching users (e.g. patients, healthcare providers, medical service vendors, pharmacies, insurance providers etc.) based on needs, via transactions on AI empowered non-blockchain-based network, or a blockchain-based network, as described herein. One or more aspects of the apparatus include at least one processor; at least one memory storing instructions executable by the at least one processor; a public blockchain component configured to provide public information via a public access layer of a blockchain-based distributed computer network in response to a public request for information about a user; and a privileged blockchain component configured to provide private information via a privileged access layer of the blockchain-based distributed computer network in response to a verified request for information about the user.

Some examples of the apparatus, system, and method further include a smart contract component configured to create and execute a smart contract, wherein the smart contract operates on the blockchain-based distributed computer network. In some aspects, the smart contract includes instructions configured to compute a plurality of component rating factors using a plurality of machine learning models; and compute the plurality of component rating factors using an ensemble algorithm to obtain a matching prediction related to a user, a healthcare provider, a diagnosis, and/or treatment plan. In some aspects, the smart contract includes instructions configured to perform a private transaction via the privileged access layer of the blockchain-based distributed computer network, wherein the private transaction suggests one or more of a healthcare provider, a diagnosis, and/or a treatment plan to a user.

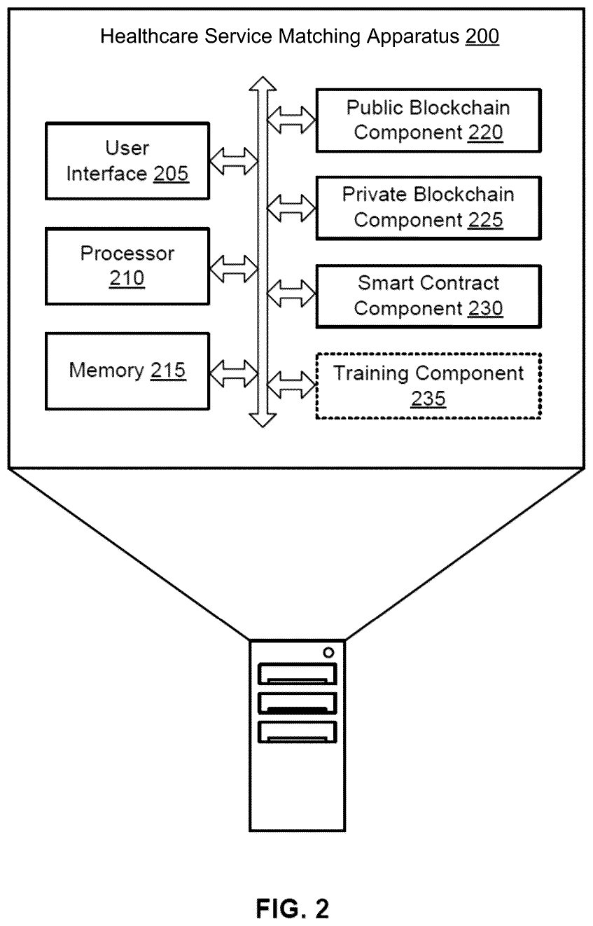

FIG. 1 shows an example of a healthcare matching system according to aspects of the present disclosure. The example shown includes a healthcare matching apparatus 100, database 105, network 110, and user interface 115. Healthcare matching apparatus 100 is an example of, or includes aspects of, the corresponding element described with reference to FIG. 2. User interface 115 is an example of, or includes aspects of, the corresponding element described with reference to FIG. 2. For example, user interface 115 may be implemented on an edge user device as shown in FIG. 1, or may be implemented directly on the healthcare matching apparatus 100 as shown in FIG. 1.

In an example, a user provides a healthcare information request via user interface 115. The information request may be a request for information about a specific healthcare professional, or generally to find a relevant healthcare professional, diagnosis, and/or treatment plan. Heatlhcare matching apparatus 100 processes the request received via the user interface. In some cases (e.g., where the request requires accessing privileged healthcare data), the processing may include verifying a permission levels of the user submitting the request. The processing may further include utilizing a smart contract to automatically perform sub-tasks related to the request. The sub-tasks may involve retrieving information from database 105. In some cases, the database 105 is an index database of data from a blockchain. In some cases, operations with database 105 refers to operations with the blockchain directly. The healthcare matching apparatus 100 then provides a result of the request for information to the user via user interface 115.

Embodiments of healthcare matching apparatus 100 include hardware and/or software components that are implemented on a server. A server provides one or more functions to users linked by way of one or more of the various networks. In some cases, the server includes a single microprocessor board, which includes a microprocessor responsible for controlling all aspects of the server. In some cases, a server uses the microprocessor and protocols to exchange data with other devices and/or users on one or more of the networks via protocols such as (but not limited to) hypertext transfer protocol (HTTP), simple mail transfer protocol (SMTP), file transfer protocol (FTP), simple network management protocol (SNMP), and/or the like. In some cases, a server may be configured to send and/or receive hypertext markup language (HTML) formatted files (e.g., for displaying web pages). In various embodiments, a server may comprise or be embodied as a general-purpose computing device, a personal computer, a laptop computer, a mainframe computer, a supercomputer, or any other suitable processing apparatus.

Database 105 is configured to store information used by the healthcare apparatus 100, including (but not limited to) indexed data related to healthcare providers, diagnoses, treatment plans, etc. from a blockchain, machine learning model parameters, cached code or boilerplate used in smart contract generation, user parameters, etc. In some embodiments, the data stored by the database 105 may include data from one or more Electronic Health Records (EHRs) and/or Electronic Medical Records (EMRs), survey data from one or more patients, insurance claims data, data related to a patient illness (e.g., pictures and/or video, symptom descriptions, other illness data), etc. A database is an organized collection of data. For example, a database stores data in a specified format known as a schema. A database may be structured as a single database, a distributed database, multiple distributed databases, or an emergency backup database. In some cases, a database controller may manage data storage and processing in a database. In some cases, a user interacts with a database controller. In other cases, a database controller may operate automatically without user interaction. As used herein, database 105 may also directly refer to a distributed blockchain structure. According to some aspects, an offline database may interact with data that is stored “on chain.” For example, one or more identifiers may be stored on chain, and corresponded with an offline database to enable additional data storage. Database 105 may implement such a combined system.

Network 110 facilitates the transfer of information between the healthcare matching apparatus 100, the database 105, and a user, e.g. through the user interface 115. In some cases, network 110 may be referred to as a “cloud”. A cloud is a computer network configured to provide on-demand availability of computer system resources, such as data storage and/or computing power. In some examples, the cloud provides resources without active management by the user. The term “cloud” is sometimes used to describe data centers available to many users over the Internet. Some large cloud networks have functions distributed over multiple locations from central servers. A server is designated an edge server if it has a direct or close connection to a user. In some cases, a cloud is limited to a single organization. In other examples, the cloud is available to many organizations. In one example, a cloud includes a multi-layer communications network comprising multiple edge routers and core routers. In another example, a cloud is based on a local collection of switches in a single physical location. A cloud may also refer to a distribution of nodes that store a blockchain and perform operations thereon.

User interface 115 enables a user to interact with a device. In some embodiments, the user interface 115 may include an audio device, such as an external speaker system, an external display device such as a display screen, or an input device (e.g., remote control device interfaced with the user interface 115 directly or through an I/O controller module). In some cases, a user interface 115 may be a graphical user interface 115 (GUI).

FIG. 2 shows an example of a healthcare matching apparatus 200 according to aspects of the present disclosure. The example shown includes healthcare matching apparatus 200, user interface 205, processor 210, memory 215, public blockchain component 220, private blockchain component 225, smart contract component 230, and training component 235. The healthcare matching apparatus 200 may be an example of, or may include aspects of, the corresponding element described with reference to FIG. 1. User interface 205 is an example of, or includes aspects of, the corresponding element described with reference to FIG. 1.

Embodiments of the healthcare matching apparatus 200 include several components and sub-components. These components are variously named and are described so as to partition the functionality enabled by the processor(s) and the executable instructions included in the computing device used in real estate matching apparatus 200 (such as the computing device described with reference to FIG. 12). In some examples, the partitions are implemented physically, such as through the use of separate circuits or processors for each component. In some examples, the partitions are implemented logically via the architecture of the code executable by the processors.

Embodiments of the healthcare matching apparatus are configured to interface with and perform operations on a blockchain. A blockchain is a distributed ledger technology that is used to record transactions and data in a way that is secure, transparent, and verifiable. It includes a decentralized network of computers, or nodes, which are connected over the internet and work together to validate and record transactions on a shared digital ledger. Each transaction that is recorded on the blockchain is added to a block, which is then cryptographically linked to the previous block in the chain. This creates a permanent and unchangeable record of all the transactions that have taken place on the blockchain.

One of the key features of some blockchains is that the blockchain is decentralized, meaning that the blockchain is not controlled by a single entity or organization. Instead, the blockchain is maintained by a network of participating nodes, which work together to validate and record transactions on the ledger. This makes the blockchain resistant to tampering and helps to ensure that the information recorded on the blockchain is accurate and trustworthy. Blockchains may be used in a variety of applications, including cryptocurrency, supply chain management, and voting systems. They are known for their security, transparency, and ability to create a permanent and verifiable record of transactions. In this usage case, every user/patient can be a node, and each node may control the information in the node with its access, and can interact via one or more smart contracts (which may optionally use artificial intelligence for at least a portion of their processing power) with a healthcare professional, a healthcare vendor, a treatment plan, and/or any other node also on the blockchain, to run necessary healthcare procedure.

Public blockchain component 220 interfaces with a public layer of the blockchain. When a user makes a public request for information, the public blockchain component 220 queries the public layer of the blockchain for the information and returns the retrieved information. The public blockchain component 220 may prevent unauthorized users from accessing information from privileged layers of the blockchain. That is, the public blockchain component 220 may be prevented from accessing a privileged or private layer of the blockchain. In this way, the blockchain can help to ensure that public queries do not gain access to information stored in private layers of the blockchain. Similarly, a private blockchain component 225 interfaces with a private layer of the blockchain. When a user makes a verified request for information, the private blockchain component 225 queries a corresponding privileged layer of the blockchain for the information and returns the retrieved information.

Computing systems (e.g., networks) may implement security measures to prevent unauthorized users (e.g., devices) from accessing system resources such as system information, data, hardware, software, applications, etc. For instance, computing systems may employ authentication procedures to authenticate a user (e.g., confirm a user's claimed identity) prior to granting the user access to restricted system resources. In an authentication procedure, a user may provide one or more credentials that may be authenticated by the computing system for the user to gain access to system resources. For example, a user may provide credentials such as a username, a password, a gesture, a biometric signature (e.g., a fingerprint), a personal identification number (PIN), processing fees associated with privileged blockchain access, etc. The computing system may compare credentials provided by the user with previously established credentials associated with the user to determine whether to permit or deny access requested by the user (e.g., where the previously established credentials may be registered with the computing system prior to the authentication procedure).

According to some aspects, the private blockchain component 225 may provide, via one or more smart contracts, the private information via a privileged access layer of the blockchain-based distributed computer network in response to a verified request for information about the real estate property. In some examples, private blockchain component 225 performs, by the one or more smart contract, a private transaction via the privileged access layer of the blockchain-based distributed computer network, where the private transaction transfers a user's private healthcare information to run a healthcare service process.

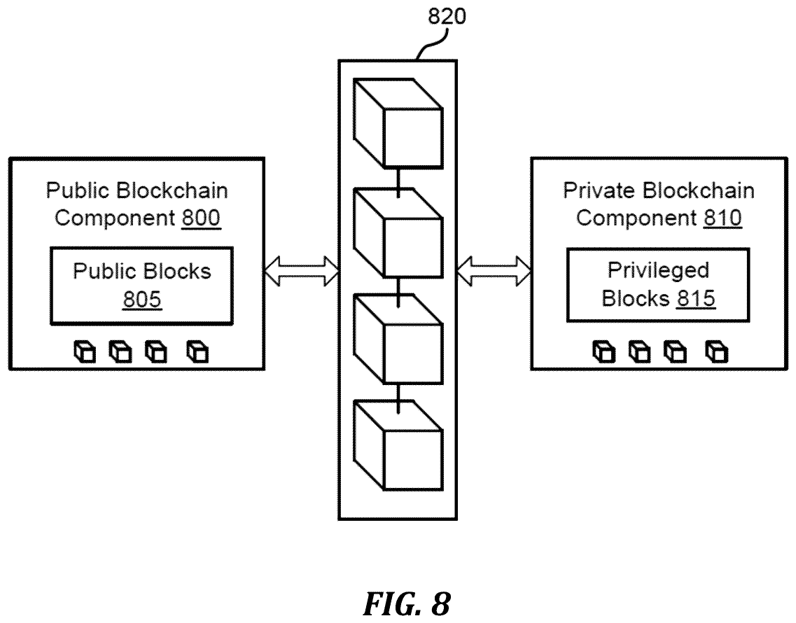

In some examples, the private blockchain component 225 receives, via the one or more smart contracts, a first verification from a first user regarding the privileged access layer of the blockchain-based distributed computer network. In some examples, private blockchain component 225 receives, via the one or more smart contracts, a second verification from a second user regarding the privileged access layer of the blockchain-based distributed computer network, where the private transaction is performed based on the first verification and the second verification. In some examples, private blockchain component 225 transfers, using the one or more smart contracts, a monetary value via the privileged access layer of the blockchain-based distributed computer network in response to a verified transaction. In some examples, private blockchain component 225 identifies a sender of the verified request. In some examples, private blockchain component 225 verifies an authorization of the sender, where the transfer of private information is based at least in part on the authorization. The private blockchain component 225 is an example of, or includes aspects of, the corresponding element described with reference to FIG. 8. Similarly, the public blockchain component 220 is an example of, or includes aspects of, the corresponding element described with reference to FIG. 8.

Smart contract component 230 may be configured to generate one or more smart contracts, including (but not limited to) code that is interpretable and/or executable by the blockchain. Embodiments of smart contract component 230 may include one or more machine learning (ML) models to enable the “AI-powered” smart contracts referenced herein. Embodiments of smart contract component 230 include a match generation model to generate matches of a patient with a healthcare professional, medical institution, insurance, pharmacy, other medical service vendor, and/or the like; an image/video information extraction model to extract illness information for diagnosis based on patient provided image or video; a diagnosis match generation model to generate illness diagnosis based on information extracted from patient image/video; a treatment plan matching model to generate a treatment plan (e.g., including a medication) based on the diagnosis; a compliance classification model to help ensure the generated information is in compliance with various regulation (e.g., HIPAA); and/or combinations thereof.

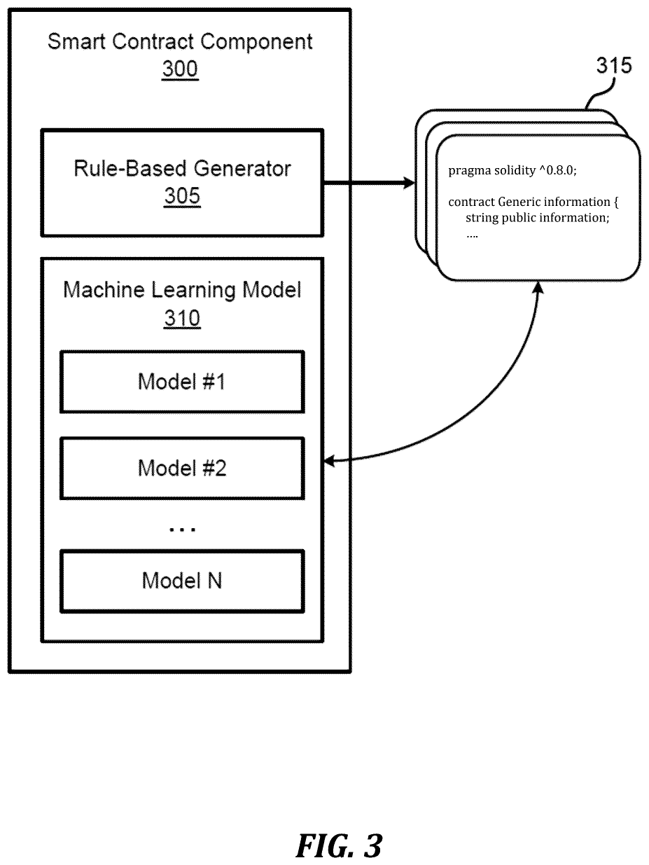

According to some aspects, the smart contract component 230 is configured to create and execute a smart contract, where the smart contract operates on the blockchain-based distributed computer network. In some aspects, the smart contract may include instructions configured to compute a set of component rating factors using a set of one or more machine learning models; and compute the set of component rating factors using an algorithm (e.g., an ensemble algorithm) to obtain a healthcare process matching prediction. In some aspects, the smart contract may include instructions configured to perform a private transaction via the privileged access layer of the blockchain-based distributed computer network, where the private transaction performs (or indicates performance of) a private healthcare service. Smart contract component 230 is an example of, or includes aspects of, the corresponding element described with reference to FIG. 3.

Training component 235 is configured to set and/or update parameters of the AI/ML models of smart contract component 230. Training component 235 may train the models in a pre-training phase and update the models in a fine-tuning phase. In some embodiments, training component 235 may continually or periodically update the models as the system is used. For example, according to some aspects, training component 235 identifies a transaction or service run on the blockchain-based distributed computer network and updates the machine learning model(s) based on the transaction.

FIG. 3 shows an example of a pipeline for smart contract 315 creation according to aspects of the present disclosure. The example shown includes smart contract component 300 and smart contract 315. In one aspect, smart contract component 300 includes rule-based generator 305 and machine learning model 310.

Smart contract component 300 may include a rule-based generator 305 used to generate smart contracts. For example, the rule-based generator 305 may include one or more template smart contract documents. The rule-based generator 305 may place the code of the machine learning model 310 and/or the output generated by the machine learning model 310 to generate the smart contract. In at least one embodiment, a non-rule-based model may be used to construct the smart contracts; for example, a trained artificial neural network (ANN) model may be used to generate a template, and then additions to the template may be made using meta-programming techniques.

Machine learning model 310 includes one or more trained models. Machine learning model 310 may be included or be based on one or more artificial neural networks (ANNs). An ANN is a hardware and/or software component that may include a number of connected nodes (artificial neurons), which loosely correspond to (e.g., function similarly to, are arranged similarly to) the neurons in a human brain. Each connection, or edge, may be capable of transmitting a signal from one node to another (like the physical synapses in a brain). Responsive to a node receiving a signal, the node may be configured to process the signal and transmit the processed signal to one or more other connected nodes. In some cases, the signals between nodes comprise real numbers, and the output of each node is computed by a function of its inputs. In some examples, nodes may determine their output using other mathematical algorithms (e.g., selecting the maximum or minimum of the inputs as the output, computing an average of the inputs, computing a difference between the inputs, and/or any other mathematical or algorithmic process using the inputs) or any other suitable algorithm for activating the node.

Each node and/or edge may be associated with one or more node weights that are used to determine how the signal is processed and transmitted. During the training process, these weights may be adjusted to improve the accuracy of the result (e.g., by lowering or minimizing a loss function which corresponds in some way to the difference between the current result and the target result). The weight assigned to an edge increases or decreases the strength of the signal transmitted between nodes along that edge. In some cases, a node may have a threshold below which a signal is not transmitted at all. In some examples, the nodes may be aggregated into layers. Different layers perform one or more different transformations on their inputs. The initial layer is known as the input layer and the last layer is known as the output layer. In some cases, signals traverse one or more layers of the model multiple times.

One or more of the AI/ML models included in the machine learning model 310 may be a natural language generation (NLG) model. NLG models are configured to generate natural language or other parseable sequences, such as code, JSON data, etc. NLG models may include transformer network architecture configured to perform an attention operation.

A transformer network is a type of neural network model which may be particularly useful for healthcare data processing tasks. The transformer network may transform one sequence of healthcare data into another sequence using an encoder and/or a decoder. One or more (e.g., both) of the encoder or the decoder may include multiple modules that can be stacked together. The included modules may include multi-head attention and/or feed-forward layers. The inputs and outputs, such as medical records, treatment plans, etc., may be embedded into an n-dimensional space. In some cases, positional encoding may be added to the embedded representation of each word and/or data point to maintain the order of the sequence elements, as the sequence depends on their order.

As a particular example, the machine learning model 310 may include a multimodal transformer model. The multimodal transformer model may process and/or fuse information from various data modalities, such as structured electronic health records (EHRs), unstructured clinical notes, medical images, and patient-reported outcomes (PROs). The transformer model, such as BERT (Bidirectional Encoder Representations from Transformers) or its variants, may be pre-trained on a large corpus of medical literature and fine-tuned on task-specific datasets.

Data preprocessing is the process of evaluating, filtering, manipulating, and/or encoding data so that a machine learning algorithm can understand it and use the resulting output. Goals of data preprocessing include eliminating data issues (e.g., missing values), improving data quality, and making the data useful for machine learning purposes.

In preprocessing, structured data from EHRs may extracted from electronic health record systems. The structured data may include as non-limiting examples, demographics, diagnoses, procedures, and medications. The extracted data may be normalized, using data normalization techniques, such as min-max scaling or z-score normalization. Such normalization may help to ensure consistent ranges and distributions across different features. Missing values in the extracted data may be handled using various strategies such as mean imputation, median imputation, forward-filling, and/or the like, based at least in part on the temporal nature of the data. Inconsistencies and outliers within the data may be identified and addressed using statistical methods and/or domain-specific rules.

Unstructured text data (e.g., from clinical notes, such as physician notes, discharge summaries, and radiology reports), may also undergo preprocessing. The preprocessing of unstructured data may involve using natural language processing (NLP) techniques. For example, Tokenization may be performed to break down the unstructured text into individual words or subwords, enabling further analysis. Named entity recognition (NER) models, such as (but not limited to) BioBERT and/or ClinicalBERT, may be employed to identify and extract relevant medical entities, including medications, symptoms, diagnoses, and procedures. Relation extraction techniques, such as dependency parsing or co-reference resolution, may be applied to identify relationships between entities and capture more complex information from the unstructured text.

Preprocessing may include use of Convolutional Neural Networks (CNNs) to extract visual features from various medical imaging modalities, such as X-rays, CT scans, MRIs, ultrasound images, and/or the like. Transfer learning approaches, such as using pre-trained CNNs like ResNet or DenseNet, may be employed to leverage learned features from large-scale image datasets. Data augmentation techniques, such as rotation, flipping, and/or elastic deformations, may be applied to increase the diversity and robustness of the image dataset. Segmentation models, such as U-Net and/or Mask R-CNN, may be used to identify and localize specific regions of interest within the medical images.

Preprocessing of time series data (e.g., Electrocardiogram (ECG) and/or electroencephalogram (EEG)) signals may include extracting meaningful features and patterns from the data. Signal preprocessing techniques, such as (but not limited to) filtering, detrending, and/or normalization may be applied to remove noise, baseline wander, and artifacts, among other things. Time-frequency analysis methods, such as short-time Fourier transform (STFT) and/or wavelet transform, may be used to capture temporal and/or spectral information from the signals. Segmentation and feature extraction techniques (e.g., R-peak detection for ECG, spectral power analysis for EEG) may be employed to derive relevant features for additional analysis.

Preprocessing of Patient-Reported Outcomes (PROs) may include the use of standardized questionnaires, such as quality of life assessments or symptom severity scales, to collect patient-reported data. Responses to the questionnaires may encoded into numerical representations, such as binary indicators or Likert scale values. Text mining techniques (e.g., sentiment analysis, topic modeling, etc.) may be applied to open-ended responses to extract additional insights.

Multimodal Fusion involves combining (“fusing”) the preprocessed data from different modalities, using techniques including (but not limited to) concatenation, attention mechanisms, and/or cross-modal attention. The fused representations are passed through transformer layers to capture the interactions and dependencies among the multimodal features.

The machine learning model 310 may be used to provide Patient provider Matching. In Patient-Provider Matching, fused patient representations may include multimodal patient data, such as EHRs, clinical notes, medical images, time series data, and PROs related to the patient, creates a comprehensive patient representation. Techniques such as concatenation, attention mechanisms, and/or cross-modal attention may be employed to effectively combine the features from different modalities. The fused representation may be used to capture the patient's overall health status, medical history, and specific needs.

Healthcare provider profiles may be constructed based on various attributes, including (but not limited to) the specialties, experience, education, publications, and patient ratings of the provider. Specialties may be encoded using medical ontologies and/or hierarchical classification schemes to capture the relationships between different areas of expertise. Experience may be quantified based on various factors, including years of practice, number of procedures performed, case volume in specific domains, and/or the like. Patient ratings and feedback may be aggregated and normalized to provide a measure of doctor quality and patient satisfaction.

Compatibility Scoring between the patient and the healthcare provider may be calculated using, as one example, cosine similarity. Additionally or alternatively, other distance metrics, such as Euclidean distance, Mahalanobis distance, or Jaccard similarity, maybe employed, depending on the nature of the data and the desired properties of the matching algorithm. The compatibility score may take into account the similarity between the patient's needs and the doctor's expertise, as well as the alignment between the patient's preferences and the doctor's communication style and bedside manner.

In some embodiments, a predetermined number of healthcare providers may be recommended. For example, the top-k most compatible doctors may be recommended to each patient based on their specific needs and preferences. Alternatively, all providers having a compatibility score with the patient that exceeds a threshold value may be recommended. The recommendation algorithm may consider various factors, such as the patient's location, insurance coverage, and preferred language to help ensure practical feasibility of the provided matches. Collaborative filtering techniques, such as matrix factorization and/or neighborhood-based methods, may be applied to leverage the preferences and experiences of similar patients when generating recommendations. Additionally or alternatively, the recommendations may take into account contextual factors, such as the patient's current health status, urgency of the medical condition, and/or availability of healthcare providers, to provide timely and relevant recommendations.

In some embodiments, the model 310 may be used to provide explanations and/or insights into factors that influenced the matching process, promoting transparency and trust. Feature importance techniques e.g., (SHAP (SHapley Additive exPlanations), LIME (Local Interpretable Model-agnostic Explanations), etc.) may be employed to highlight the key attributes that contributed to the compatibility scores. Visual explanations, such as heat maps or attention visualizations, may be used to illustrate the salient regions or features in medical images or time series data that influenced the matching decision.

The machine learning model 310 may allow patients to provide feedback regarding the recommended healthcare providers, capturing the experiences and/or satisfaction levels of the patient. Feedback mechanisms (e.g., ratings, reviews, questionnaires, etc.) may be incorporated to gather patient insights and preferences. The collected feedback may be used to refine the matching algorithm of the model 310, update doctor profiles, and/or otherwise improve future recommendations (e.g., for other patients, and/or for the reviewing patient) through continuous learning and adaptation.

The machine learning model 310 may be used for diagnosis and treatment recommendation. A multimodal patient representation may be provided as input into a trained classification module to predict most likely diagnoses based on the patient's history and current symptoms. In some embodiments, a knowledge graph including disease-symptom relationships, drug-drug interactions, and/or treatment guidelines may be incorporated to provide evidence-based recommendations for the diagnosis and/or the suggested treatment. The machine learning model 310 nay generate personalized treatment plans, considering the patient's specific conditions, allergies, and preferences.

A transformer network may incorporate an attention mechanism. The attention mechanism may examine an input sequence and determine which parts of the sequence are significant at each step. For example, the attention mechanism may involve queries, keys, and/or values, represented as Q, K, and V respectively. Q may be a matrix containing the query or the vector representation of a single word or data point in the sequence; K may include vector representations of one or more (e.g., all) words and/or data points in the sequence; and V may include a vector representations of these words or data points again. In the encoder and decoder multi-head attention modules, V may be the same sequence as Q. However, in an attention module that considers both the encoder and decoder sequences, V may differ from the sequence represented by Q. In some cases, the values in V may be multiplied and/or summed with corresponding attention weights, denoted as a, to determine the importance of each data point in the sequence. This approach is particularly useful in healthcare for tasks such as (but not limited to) patient record analysis, medical image interpretation, and/or generating treatment plan recommendations, where the ability to focus on specific parts of the data can enhance the accuracy and/or relevance of the results

In some embodiments, the machine learning model 310 may include one or more classification models. Classification models are trained on (often extensive) sets of training data. Each classification model may be designed to accurately categorize input data. For example, a classification model in healthcare may be used to analyze input patient data to identify and categorize illnesses, diagnoses, and/or treatment outcomes. In practice, a classification model may process a patient's illness or diagnosis to determine if it meets regulatory standards. This may involve training the classification model with vast amounts of data, including both compliant and non-compliant cases, allowing the classification model to learn the nuanced differences between compliant and non-compliant cases. In particular, the classification model may process an input vector that represents a patient's healthcare status. The vector may include various data points such as (but not limited to) symptoms, lab results, medical history, and/or current diagnoses. Once the input data vector is processed, the trained classification model, such as an Artificial Neural Network (ANN), uses learned patterns to predict whether the healthcare status adheres to established regulations. These regulations could pertain to treatment protocols, medication guidelines, and/or clinical procedures to be followed.

Other models within machine learning model 310 may include image and/or video processing or generative models configured to, for example, produce extracted information for a healthcare diagnosis and/or generate images to confirm a diagnosis and/or educate a user. The image and/or video data may be generated by a generative architecture such as a diffusion model. In some embodiments, the image and/or video data is generated into a vector representation that is interpretable by an image and/or video rendering component downstream.

In some embodiments, machine learning model 310 predicts a match between a patient and a health care provider, between a patient and a treatment plan, between a patient and a healthcare service provider, between a patient and a diagnosis, and/or the like, by using vector representation(s) of the patient, health care provider, treatment plan, health care service provider, and/or diagnosis as input. Some embodiments are configured or designed to search or explore an embedding space of patients and/or healthcare services to make one or more accurate match predictions.

In certain implementations, the machine learning model 310 generates a healthcare service matching prediction in response to a verified request. This prediction may encompass a ranked list of one or more healthcare providers, service vendors, diagnoses, treatments, medications, and/or pharmacies. The machine learning model 310 operates by calculating a set of component rating factors using various machine learning models within its framework. The model then integrates these component rating factors using an ensemble algorithm to derive the final healthcare service matching prediction. This ensemble method enhances the prediction accuracy by leveraging the strengths of multiple models. Furthermore, the machine learning model 310 can identify a specific diagnosis, treatment, or provider/vendor based on the healthcare process matching prediction. This identification process ensures that the private information is tailored and provided to the patient in accordance with the verified request. By utilizing advanced algorithms and comprehensive data processing, the model helps to ensure precise and reliable healthcare service matching.

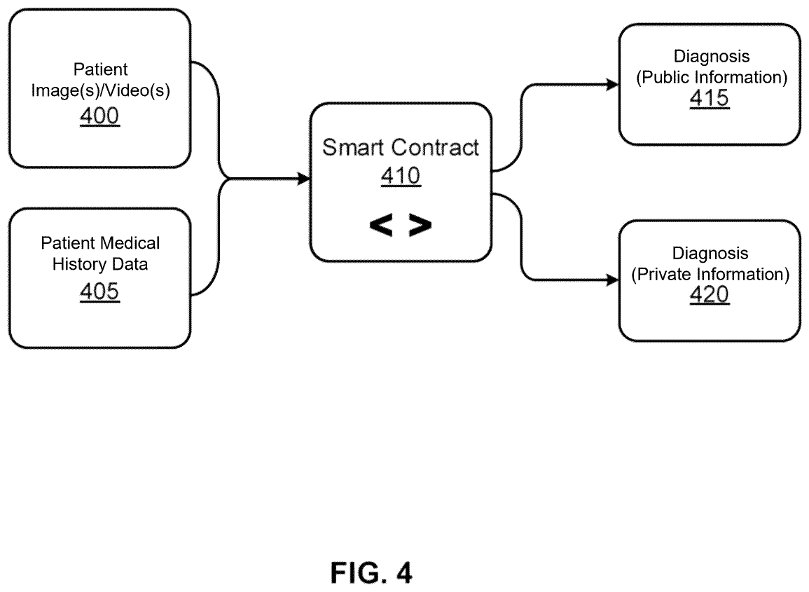

In some embodiments, smart contract component 300 generates a smart contract 315 including code executable by a blockchain to perform the functionality described above. Some embodiments of smart contract component 300 include one or more models configured to generate one or more rating factors for use in a smart matching process. Smart contract component 300 is an example of, or includes aspects of, the corresponding element described with reference to FIG. 2. Smart contract 315 is an example of, or includes aspects of, the corresponding element described with reference to FIGS. 4-7.

Some examples of smart contract 315 are configured to manage and enforce access rights and privileges for different levels of users. For example, some users (e.g., non-selected healthcare providers, vendors, etc.) are granted visibility to only basic patient healthcare information, whereas specific users (e.g., selected healthcare providers, vendors, etc.) may have access to more comprehensive information. Higher-level vendors may have access to premium information. For example, in some cases, certain healthcare providers and medical service vendors may have the most extensive access, including private-only information, responsive to the patient granting access to that provider or vendor. In some cases, details are available exclusively to patients and/or healthcare providers who provide a specific service (e.g., a service related to the details).

Smart contracts such as smart contract 315 facilitate healthcare service by processing private data within secure private layers of the blockchain. This approach includes managing the flow of healthcare services, such as (but not limited to) diagnosis treatment, medication, and review of treatment results to adjust a treatment plan, where the healthcare service details may be stored in the private layer and are accessible only to authorized healthcare providers, vendors, and/or the patient him/herself. The smart contracts may connect patient and healthcare provider, vendor, diagnosis, and/or treatment/medication through a matching algorithm. For instance, the smart contract may link a patient to an appropriate physician or healthcare vendor based on the physician's access rights, and also connect patient to treatment plan or medication when the service necessitates. These smart contracts can be generated from templates and incorporate functionality from AI/ML models, enhancing the process of matching, diagnosis, treatment/medication, and/or other related healthcare services using the blockchain.

In some cases, the generated smart contracts control the creation of different views of information, tailored to the access level of each user. For example, in a GUI such as a web-portal, a healthcare provider/vendor may select different views of the patient information according to their granted level of access. The views may display different sets of information, such as premium or privileged information about a patient. Examples of premium information may include full medical history, previous treatment and recovery information, insurance provider information, potential or projected health concerns, and/or the like. The smart contracts can assign and enforce access policies for private data, allowing for the distribution of information to different healthcare provider/vendor groups with varying terminology and data requirements. The smart contracts are isolated from the data, permitting multiple policies per patient and enabling updates to policies without affecting other aspects of the system. In healthcare services, such as diagnosis and/or treatment with relative results, smart contracts may operate within the private layers, ensuring that sensitive data is processed securely and remains inaccessible to general public.

FIG. 4 shows an example of a pipeline for healthcare diagnosis processing according to aspects of the present disclosure. The example shown includes patient image/video(s) 400, patient medical history data 405, smart contract 410, diagnosis (public info.) 415, and diagnosis (private info.) 420. Smart contract 410 is an example of, or includes aspects of, the corresponding element described with reference to FIG. 3, and 5-7.

In this example, a smart contract 410 (or an AI/ML model included therein) receives input information including patient image/video(s) 400 regarding illness, and the extracted information from the image/video, patient medical history data 405. One or more models included in the smart contract 410 process the information to generate both a public info diagnosis 415 and private info diagnosis 420. For example, a classification model may be used to determine which data within the patient medical history data 405 should belong to a private or privileged access-level, and which data is suitable for the general view. An NLG model may, for example, generate additional description of the diagnosis to be included. Some examples of a public version of diagnosis data include only symptom data, whereas a private version of the diagnosis data may include the information such as an underlying illness causing the symptom, suggested treatment plans, and/or the like. In some cases, the NLG model may generate an aesthetically pleasing and naturally flowing description of the diagnosis based on a minimal input of the symptoms. In some embodiments, an image/video to text model processes the patient image/video data 400 to produce a description of the patient symptom based on uploaded image and/or video data for incorporation into the diagnosis (e.g., in place of or in addition to the image and/or video data).

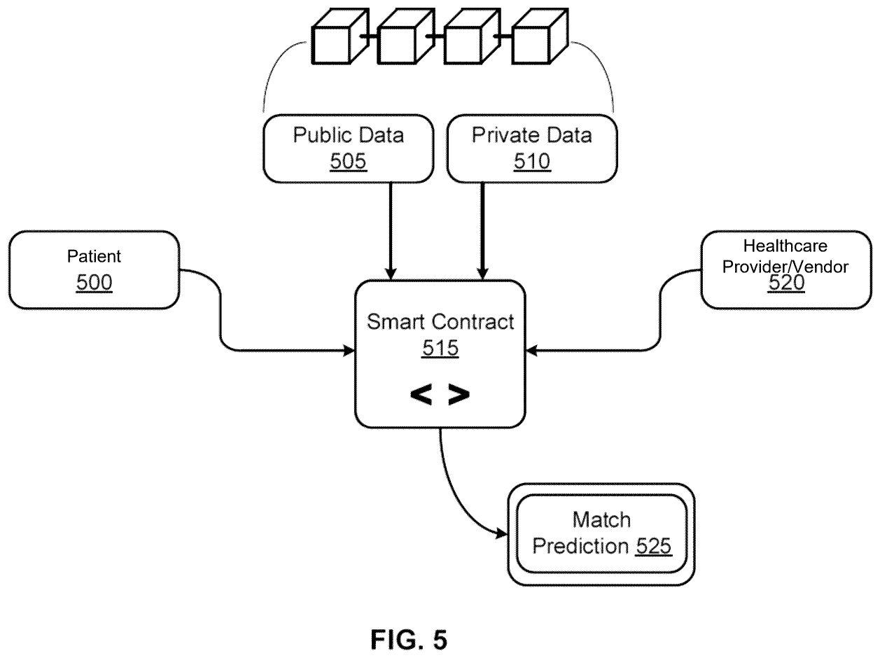

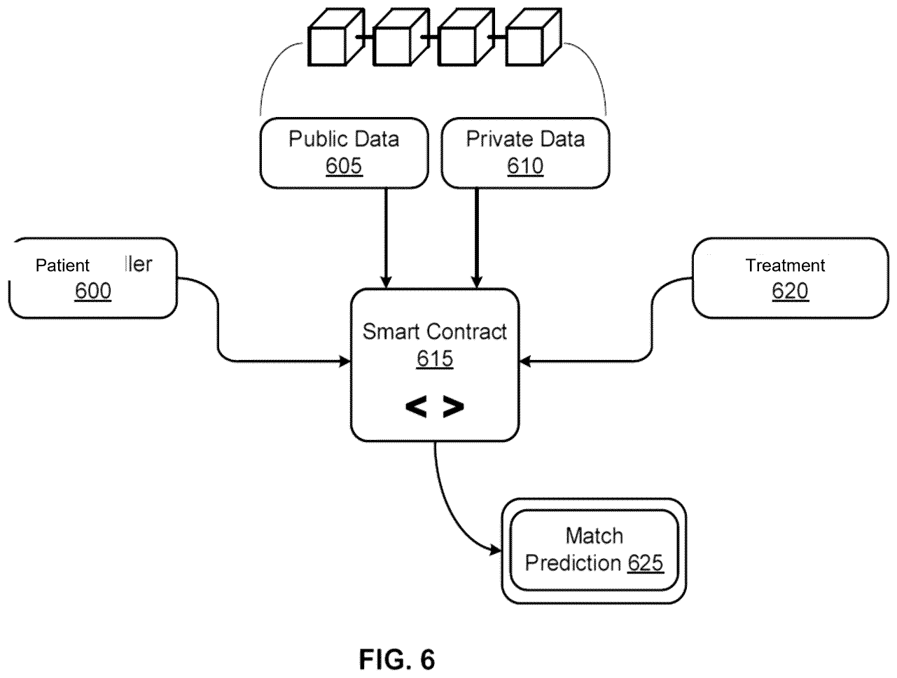

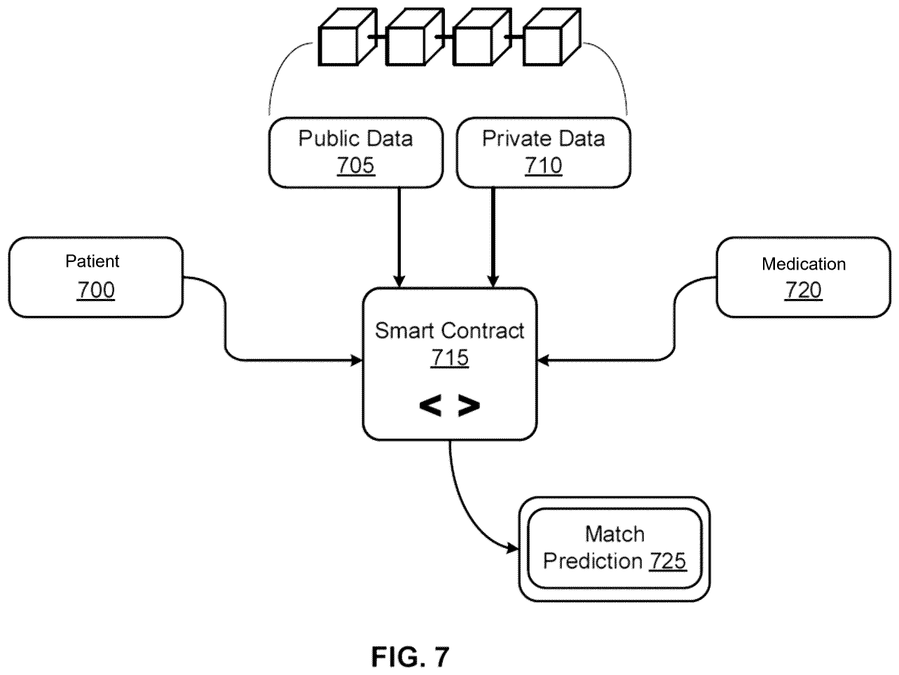

FIG. 5 shows an example of a pipeline for patient 500 to healthcare provider or vendor 520 matching according to aspects of the present disclosure. The example shown includes patient 500, patient public data 505, patient private data 510, smart contract 515, healthcare provider or vendor 520, and match prediction 525. The patient public data 505 may be an example of, or may include aspects of, the corresponding element described with reference to FIGS. 6 and 7. The patient private data 510 may be an example of, or may include aspects of, the corresponding element described with reference to FIGS. 6 and 7. Smart contract 515 is an example of, or includes aspects of, the corresponding element described with reference to FIGS. 3, 4, 6, and 7. Match prediction 525 is an example of, or includes aspects of, the corresponding element described with reference to FIGS. 6 and 7.

In this example, patient 500 queries the healthcare process matching system requesting healthcare service. The healthcare service may be provided by a healthcare provider (e.g., a physician, psychologist, physical therapist, etc.) or healthcare vendor (e.g., medical institute, pharmacy, insurance provider, etc.) 520. The smart contract 515 retrieves patient public data 505 and/or patient private data 510 to predict if the service of a particular healthcare provider or vendor 520 is a match with the patient needs. The match prediction 525, may be in the form of an output vector or scalar. According to some aspects, the smart contract 515 may compute a rating factor between patient 500 and the service of the healthcare provider or vendor 520, and then computes the match prediction 525 based on the rating factor. According to some aspects, the smart contract 515 may retrieve private data 510 only in response to a service by healthcare provider/vendor 520 or the request made by patient 500.

FIG. 6 shows an example of a pipeline for matching a patient 600 to a treatment plan according to aspects of the present disclosure. The example shown includes patient 600, patient public data 605, patient private data 610 (e.g., patient diagnosis and symptom information), smart contract 615, treatment 620, and match prediction 625. In this example, smart contract 615 may compute the match prediction 625 using the same or similar methods described with reference to FIG. 5. In some examples, smart contract 615 computes a user-user match between patient 600 and treatment 620. In other examples, smart contract 615 computes a patient-healthcare provider(service vendor) match between patient 600 and healthcare provider or service vendor (e.g., specializing in the diagnosis included in the patient private data 610) associated with the treatment 620. In some embodiments, the patient 600 queries the healthcare matching system with natural language using a prompt. Additionally or alternatively, the patient 600 queries the healthcare matching system using a user interface, such as the one described with reference to FIGS. 1-2.

Similarly, FIG. 7 shows an example of a pipeline for matching the patient with a medication according to aspects of the present disclosure. The example shown includes patient 700, patient public data 705, patient private data 710 (e.g., including patient diagnosis and/or symptom information), smart contract 715, medication 720, and match prediction 725. In this example, smart contract 715, may compute match prediction 725 using the same or similar methods described with reference to FIGS. 5-6. The patient 700 may include additional information in their request to the healthcare matching system, such as a natural language description of a latest symptom update, or some latest illness diagnosis. Examples of latest illness diagnoses, or symptom updates may include (but need not be limited to) a latest fever increase or reduction, mucus or cough improvement, or any other quantitative or qualitative change in a symptom. In this way, the matching medication (e.g., as indicated in the match prediction 725) may be adjusted according to the most up-to-date information for a more efficient cure.

FIG. 8 shows an example of public and private layers according to aspects of the present disclosure. The example shown includes public blockchain component 800, private blockchain component 810, and blockchain 820. Public blockchain component 800 is an example of, or includes aspects of, the corresponding element described with reference to FIG. 2. The private blockchain component 810 is an example of, or includes aspects of, the corresponding element described with reference to FIG. 2.

The public blockchain component 800 may interact with blockchain 820. Public blockchain component 800 may submit process runs to be added to blockchain 820, and/or may prevent unauthorized runs from being submitted to blockchain 820. A blockchain 820 is described with reference to FIG. 2. One non-limiting example of blockchain 820 is the Flow blockchain, though those of skill in the art will recognize that the methods described herein are not limited to operation thereon. In one aspect, the public blockchain component 800 may include one or more public blocks 805. The one or more public blocks 805 may include general information about a patient. For example, in the case of a patient matching to a healthcare provider or treatment, public blocks 805 may include the patient's very general information that can be available for public. In some embodiments, the public blockchain component 800 may submit runs with patient public data according to instruction from a smart contract.

The private blockchain component 810 may interact with blockchain 820. In one aspect, the private blockchain component 810 may include one or more privileged blocks 815. The one or more privileged blocks 815 may include privileged information about a patient. For example, in the case of a patient matching to a healthcare provider or treatment, the one or more privileged blocks 815 may include detailed information about the patient's latest symptoms, illness diagnosis, drug and/or allergic history, previous medical history, insurance information, previous physician's information and remarks, pharmacy info, and/or the like. In some embodiments, the private blockchain component 810 may submit runs with sensitive or privileged data according to instruction from a smart contract.

Accordingly, by separating the blocks that are used on a blockchain, embodiments implement different privileged layers onto the blockchain, thereby allowing for multi-level verification according to each user's permissions. Furthermore, smart contract functionality may be tailored to different types of available information. In at least one embodiment, a controlling user or authority includes the highest level of permissions, enabling the master user/wallet/authority to develop different automations or functionalities using data from the public and private blocks.

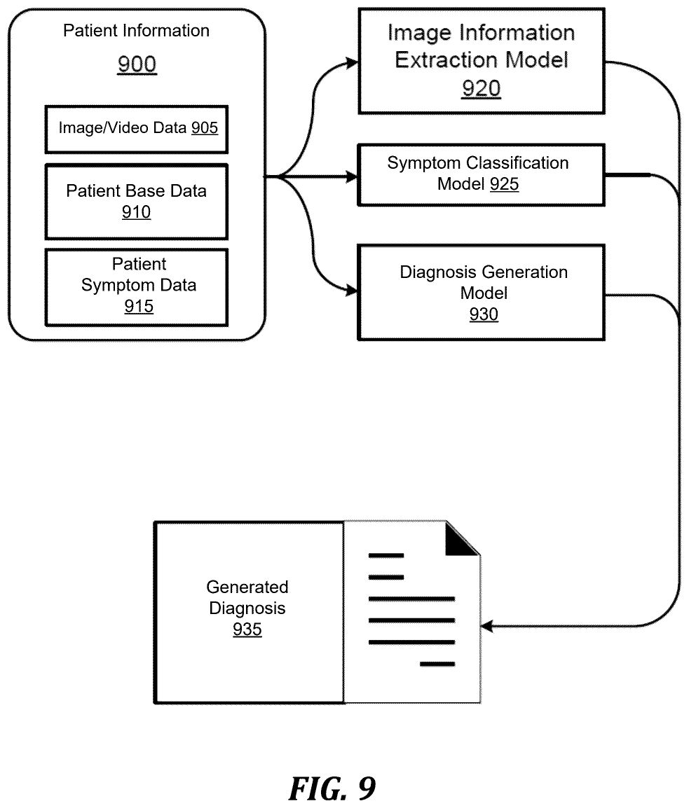

FIG. 9 shows an example of a diagnosis generation model according to aspects of the present disclosure. The example shown includes patient information 900, image information extraction model 920, symptom classification model 925, diagnosis generation model 930, and generated diagnosis result 935.

In this example, the patient information 900 includes (but need not be limited to) patient-provided image and/or video data 905 regarding an illness or symptom, patient base data 910, and patient symptom related data 915. The patient data 900 may be input into one or more AI/ML models. Each model may then transform the inputs into a patient representation that is understandable by that particular model. For example, image or video data 905 may be re-shaped into one or more tensors that the image information extraction model 920 is designed to understand.

The image information extraction model 920 may include a trained model capable of visual understanding of an image or video. Examples of such models include CLIP, BLIP, visual transformer models, and/or others. In some examples, the image and/or video information extraction model 920 may process image and/or video data 905 and output a description of the image and/or video data. This description may be provided as input to another model, such as the diagnosis generation model 930, to be expanded upon and/or tailored for the final generated diagnosis 935. Additionally, or alternatively, the description may be attached to the input image and/or video so that the description is displayed with (e.g., adjacent to, as an overlay for, or otherwise in connection with) the images in the generated diagnosis 935.

The symptom classification model 925 is configured to generate a clear symptom based on image information extracted from a natural language description of the symptom provided by the patient. The symptom classification model 925 may receive both the patient information 900, the description created by the image information extraction model 920, outputs from other models (such as the models described above), or some combination thereof.

The diagnosis generation model 930 may be configured to identify an illness or reason associated with the one or more symptoms reported by a patient. In some embodiments, the diagnosis generation model 930 may generate a natural language description of the illness or cause of the symptom(s). For example, diagnosis generation classification model 930 may receive a diagnosis generation vector including one or more symptoms reported by the patient (e.g., temperature, level of pain, color of mucus, sound of cough, and/or the like), and may identify an illness or reason for the patient's symptom(s) based on the diagnosis generation vector. Embodiments of diagnosis generation model 930 may include an ANN trained on training tuples including many diagnosis generation vector and illness pairs. The diagnosis generation and/or the training may also be based on the patient related data 915. The patient related data 915 may include, as non-limiting examples, medical information (e.g., patient medical history, previous illness and treatment), recent patient activity (e.g., travel, physical activities, etc.), environmental information (e.g., weather conditions, pollen counts, etc.), and/or other information that may be relevant to determining the diagnosis. Other representations of patient information may be used in place of or in addition to the patient-related data, including patient drug usage, family medical information, etc.

Embodiments of the diagnosis description generation model 930 may include an NLG model such as Flan-T5, GPT, and/or others. In some examples, diagnosis description generation part of 930 is based on a pre-trained language model and is then fine-tuned with additional training data.

Generated diagnosis 935 may include the diagnosed illness, as well as other features and/or information about the patient to prepare for treatment. According to some aspects, multiple versions of generated diagnosis 935 are produced, with each version being associated with a different access level of users and/or a different level of user expertise. For example, a public access level diagnosis may be different from a private access level diagnosis. Similarly, a diagnosis intended to be read and understood by a patient may be different from a diagnosis intended to be read and understood by a healthcare provider.

### Healthcare Provider, Treatment, and/or Medication Retrieval and Generation Techniques

The following depicts example methods of a plurality of methods that may be performed by at least one of the aforementioned modules, or components thereof. Various hardware components may be used at the various stages of the operations disclosed with reference to each module. For example, although methods may be described to be performed by a single computing device, it should be understood that, in some embodiments, different operations may be performed by different networked elements in operative communication with the computing device. For example, at least one computing device may be employed in the performance of some or all of the stages disclosed with regard to the methods. Similarly, an apparatus may be employed in the performance of some or all of the stages of the methods. As such, the apparatus may comprise at least those architectural components as found in computing device 1400.

Furthermore, although the stages of the following example methods are disclosed in a particular order, it should be understood that the order is disclosed for illustrative purposes only. Stages may be combined, separated, reordered, and various intermediary stages may exist. Accordingly, it should be understood that the various stages, in various embodiments, may be performed in orders that differ from the ones disclosed below. Moreover, various stages may be added or removed without altering or departing from the fundamental scope of the depicted methods and systems disclosed herein.

A method, apparatus, non-transitory computer readable medium, and/or system for automating locating and/or matching a patient to a healthcare provider, treatment plan, and/or medication using a blockchain-based network is described. In one or more aspects, the method, apparatus, non-transitory computer readable medium, and/or system may obtain, by a smart contract operating on a blockchain-based distributed computer network, both public information of a healthcare provider, treatment plan, and/or medication, and private information of the healthcare provider, treatment plan, and/or medication; provide, by the smart contract, the public information via a public access layer of the blockchain-based distributed computer network in response to a public request for information about the healthcare provider, treatment plan, and/or medication; and provide, by the smart contract, the private information via a privileged access layer of the blockchain-based distributed computer network in response to a verified request for information about the healthcare provider, treatment plan, and/or medication.

In some embodiments, the method, apparatus, non-transitory computer readable medium, and/or system may perform, by the smart contract, a private healthcare process run via the privileged access layer of the blockchain-based distributed computer network, wherein the private healthcare process runs a medical treatment for the patient.

In some embodiments, the method, apparatus, non-transitory computer readable medium, and/or system may receive, by the smart contract, a first verification from a first user via the privileged access layer of the blockchain-based distributed computer network. Some example embodiments may include receiving, by the smart contract, a second verification from a second user via the privileged access layer of the blockchain-based distributed computer network, wherein the private healthcare service run is performed based on the first verification and the second verification.

In some embodiments, the method, apparatus, non-transitory computer readable medium, and/or system may generate, by a machine learning model operating off of the blockchain-based distributed computer network, a healthcare provider, treatment, and/or medication matching prediction in response to the verified request. Some example embodiments may include providing a result of the healthcare provider, treatment, and/or medication matching prediction to the smart contract. The private information provided by the smart contract may be based, at least in part, on the result of the healthcare provider, treatment, and/or medication matching prediction. In some embodiments, the healthcare provider, treatment, and/or medication matching prediction may include a ranking of a plurality of healthcare providers, treatments, and/or medications. Some example embodiments include receiving patient information, wherein the healthcare provider, treatment, and/or medication matching prediction is based, at least in part, on the received patient information.

Some example embodiments of the method, apparatus, non-transitory computer readable medium, and/or system identify a transaction on the blockchain-based distributed computer network. Some example embodiments include updating the machine learning model based on the healthcare process runs.

Some example embodiments of the method, apparatus, non-transitory computer readable medium, and system compute a plurality of component rating factors using a plurality of machine learning models. In some embodiments, the plurality of component rating factors are combined using an ensemble algorithm to obtain the healthcare provider, treatment, and/or medication matching prediction. Some example embodiments of the method, apparatus, non-transitory computer readable medium, and/or system identify a user based on the healthcare provider, treatment, and/or medication matching prediction. The private information is provided to the identified user in response to the verified request.

In some example embodiments, the method, apparatus, non-transitory computer readable medium, and/or system may restrict access to the private information via the public access layer of the blockchain-based distributed computer network. Some example embodiments of the method, apparatus, non-transitory computer readable medium, and/or system transfer, by the smart contract, a monetary value via the privileged access layer of the blockchain-based distributed computer network in response to a verified healthcare service run.

Some example embodiments of the method, apparatus, non-transitory computer readable medium, and/or system generate, by the smart contract, feedback for use in treatment adjustment, and/or for drug further improvement and development (e.g., improvement of dosage levels to obtain desired treatment results, the substitution of one drug for another to obtain desired results, and/or medical research for a more efficacious drug). In some example embodiments, the treatment may be modified in response to the feedback, and/or the drug may be changed (e.g., in terms of dosage or substitution of one drug for another) based at least in part on the feedback. Some example embodiments of the method, apparatus, non-transitory computer readable medium, and/or system identify a sender of the verified request. The authorization of the sender may be verified, and the private information may be based (at least in part) on the authorization.

FIG. 10 shows an example of a method 1200 for retrieving privileged information according to aspects of the present disclosure. In some examples, these operations are performed by a system including a processor executing a set of codes to control functional elements of an apparatus. Additionally or alternatively, certain processes are performed using special-purpose hardware. Generally, these operations are performed according to the methods and processes described in accordance with aspects of the present disclosure. In some cases, the operations described herein are composed of various substeps, or are performed in conjunction with other operations.

At operation 1205, a user requests information. The user may be, for example, a patient, a healthcare professional, a medical institution, and/or the like. In the case of the user being a patient, the patient may request information about a healthcare professional, treatment, and/or medication; in the case of user being a healthcare professional or medical institution, the user requests information about a patient. The user may make the request via a user interface as described with reference to FIGS. 1-2. As non-limiting examples, the user may make the request by selecting a visual element from a GUI, or by typing a natural language request into a text field.

At operation 1210, the system provides public information. For example, if the user makes the request without specifying a permission level, provides incorrect or outdated authentication, fails to provide an access fee, and/or the like, the system may default to presenting public information. Moreover, if the user makes the request specifying a “public” permission level, the system may adhere to the requested permission level. Additional information regarding public information is described with reference to FIG. 3.

At operation 1215, the user provides authentication. Authentication may include logging in to a user portal with login credentials, inputting a secret key into the system, paying the access fee, and/or some combination thereof. For example, the user may provide authentication that associates the user with a healthcare service run or pending healthcare process run with a treatment identified in the request.

At operation 1220, the system verifies the authentication. For example, the system may verify the user by comparing their online profile with a wallet on the blockchain. In some embodiments, verification may include comparing the login credentials from operation 1215 to a list of approved credentials, comparing an input key to a list of approved keys, and/or verifying success of payment of an access fee.

At operation 1225, the system provides the privileged information based on the verified authentication. The access permissions of the user may be managed by a public blockchain component and a private blockchain component, along with one or more smart contracts, as described in the pipelines illustrated with reference to FIGS. 3-9.

FIG. 11 shows an example of a method 1300 for automated healthcare provider, treatment, and/or medication matching and generation according to aspects of the present disclosure. In some examples, these operations are performed by a system including a processor executing a set of codes to control functional elements of an apparatus. Additionally or alternatively, certain processes are performed using special-purpose hardware. Generally, these operations are performed according to the methods and processes described in accordance with aspects of the present disclosure. In some cases, the operations described herein are composed of various substeps or are performed in conjunction with other operations.

At operation 1305, the system obtains, by a smart contract operating on a blockchain-based distributed computer network, information for a healthcare provider, treatment, and/or medication. The obtained information may include public information and/or private information. In some embodiments, operation 1305 may refer to, or may be performed by, one or more of a public blockchain component and/or a private blockchain component of a healthcare service run matching apparatus as described with reference to FIG. 2. Both the public information and the private information may be stored on a distributed ledger system, such as a blockchain-based computer network.

At operation 1310, the system may provide, via the smart contract, the public information via a public access layer of the blockchain-based distributed computer network in response to a public request for information about the healthcare service run. In some cases, the operations of this step refer to, or may be performed by, a public blockchain component as described with reference to FIGS. 2 and 8. The information may be displayed via a user interface.

At operation 1315, the system provides, by the smart contract, the private information via a privileged or private access layer of the blockchain-based distributed computer network in response to a verified request for information about the healthcare service. In some cases, the operations of this step refer to, or may be performed by, a private blockchain component as described with reference to FIGS. 2 and 8. Additional information regarding different access levels of a user is provided with reference to FIGS. 2-3 and FIG. 10.

Embodiments of the present disclosure provide a hardware and software platform operative as a distributed system of modules and computing elements.

The platform may be embodied as, for example, but not be limited to, a website, a web application, a desktop application, a backend application, and a mobile application compatible with a computing device 1400. The computing device 1400 may comprise, but not be limited to, one or more of the following:
A mobile computing device, such as, but is not limited to, a laptop, a tablet, a smartphone, a drone, a wearable, an embedded device, a handheld device, an Arduino, an industrial device, or a remotely operable recording device;
A supercomputer, an exascale supercomputer, a mainframe, or a quantum computer;
A minicomputer, wherein the minicomputer computing device comprises, but is not limited to, an IBM AS400/iSeries/System I, A DEC VAX/PDP, an HP3000, a Honeywell-Bull DPS, a Texas Instruments TI-990, or a Wang Laboratories VS Series; and/or
A microcomputer, wherein the microcomputer computing device comprises, but is not limited to, a server, wherein a server may be rack-mounted, a workstation, an industrial device, a raspberry pi, a desktop, or an embedded device.

The platform may be hosted on a centralized server or a cloud computing service. Although the example methods 1200, 1300 may be performed by a computing device 1400, it should be understood that, in some embodiments, different operations may be performed by a plurality of the computing devices 1400 in operative communication on at least one network.

FIG. 12 shows an example of a computing device 1400 according to aspects of the present disclosure. The example shown includes computing device 1400, processor(s), memory subsystem 1410, communication interface 1415, I/O interface 1420, user interface component(s), and channel 1430.

In some embodiments, computing device 1400 is an example of, or includes aspects of, a healthcare service matching apparatus illustrated in FIGS. 1-2. In some embodiments, computing device 1400 includes one or more processors 1405 that can execute instructions stored in memory subsystem 1410 to obtain public information for a healthcare service and private information for the healthcare service; provide the public information via a public access layer of a blockchain-based distributed computer network in response to a public request for information about the healthcare service run; and provide the private information via a privileged access layer of the blockchain-based distributed computer network in response to a verified request for information about the healthcare service run.

According to some aspects, computing device 1400 includes one or more processors 1405. In some cases, a processor is an intelligent hardware device, (e.g., a general-purpose processing component, a digital signal processor (DSP), a central processing unit (CPU), a graphics processing unit (GPU), a microcontroller, an application specific integrated circuit (ASIC), a field programmable gate array (FPGA), a programmable logic device, a discrete gate or transistor logic component, a discrete hardware component, or a combination thereof. In some cases, a processor is configured to operate a memory array using a memory controller. In other cases, a memory controller is integrated into a processor. In some cases, a processor is configured to execute computer-readable instructions stored in a memory to perform various functions. In some embodiments, a processor includes special purpose components for modem processing, baseband processing, digital signal processing, or transmission processing.

According to some aspects, memory subsystem 1410 includes one or more memory devices. Examples of a memory device include random access memory (RAM), read-only memory (ROM), or a hard disk. Examples of memory devices include solid state memory and a hard disk drive. In some examples, memory is used to store computer-readable, computer-executable software including instructions that, when executed, cause a processor to perform various functions described herein. In some cases, the memory contains, among other things, a basic input/output system (BIOS) which controls basic hardware or software operation such as the interaction with peripheral components or devices. In some cases, a memory controller operates memory cells. For example, the memory controller can include a row decoder, column decoder, or both. In some cases, memory cells within a memory store information in the form of a logical state.

According to some aspects, communication interface 1415 operates at a boundary between communicating entities (such as computing device 1400, one or more user devices, a cloud, and one or more databases) and channel 1430 and can record and process communications. In some cases, communication interface 1415 is provided to enable a processing system coupled to a transceiver (e.g., a transmitter and/or a receiver). In some examples, the transceiver is configured to transmit (or send) and receive signals for a communications device via an antenna.

According to some aspects, I/O interface 1420 is controlled by an I/O controller to manage input and output signals for computing device 1400. In some cases, I/O interface 1420 manages peripherals not integrated into computing device 1400. In some cases, I/O interface 1420 represents a physical connection or port to an external peripheral. In some cases, the I/O controller uses an operating system such as iOS®, ANDROID®, MS-DOS®, MS-WINDOWS®, OS/2®, UNIX®, LINUX®, or other known operating system. In some cases, the I/O controller represents or interacts with a modem, a keyboard, a mouse, a touchscreen, or a similar device. In some cases, the I/O controller is implemented as a component of a processor. In some cases, a user interacts with a device via I/O interface 1420 or via hardware components controlled by the I/O controller.

According to some aspects, user interface component(s) 1425 enable a user to interact with computing device 1400. In some cases, user interface component(s) 1425 include an audio device, such as an external speaker system, an external display device such as a display screen, an input device (e.g., a remote-control device interfaced with a user interface directly or through the I/O controller), or a combination thereof. In some cases, user interface component(s) 1425 include a GUI.

The description and drawings described herein represent example configurations and do not represent all the implementations within the scope of the claims. For example, the operations and steps may be rearranged, combined or otherwise modified. Also, structures and devices may be represented in the form of block diagrams to represent the relationship between components and avoid obscuring the described concepts. Similar components or features may have the same name but may have different reference numbers corresponding to different figures.

Some modifications to the disclosure may be readily apparent to those skilled in the art, and the principles defined herein may be applied to other variations without departing from the scope of the disclosure. Thus, the disclosure is not limited to the examples and designs described herein but is to be accorded the broadest scope consistent with the principles and novel features disclosed herein.

The described methods may be implemented or performed by devices that include a general-purpose processor, a digital signal processor (DSP), an application specific integrated circuit (ASIC), a field programmable gate array (FPGA) or other programmable logic device, discrete gate or transistor logic, discrete hardware components, or any combination thereof. A general-purpose processor may be a microprocessor, a conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration). Thus, the functions described herein may be implemented in hardware or software and may be executed by a processor, firmware, or any combination thereof. If implemented in software executed by a processor, the functions may be stored in the form of instructions or code on a computer-readable medium.

Computer-readable media includes both non-transitory computer storage media and communication media including any medium that facilitates transfer of code or data. A non-transitory storage medium may be any available medium that can be accessed by a computer. For example, non-transitory computer-readable media can comprise random access memory (RAM), read-only memory (ROM), electrically erasable programmable read-only memory (EEPROM), compact disk (CD) or other optical disk storage, magnetic disk storage, or any other non-transitory medium for carrying or storing data or code.

Also, connecting components may be properly termed computer-readable media. For example, if code or data is transmitted from a website, server, or other remote source using a coaxial cable, fiber optic cable, twisted pair, digital subscriber line (DSL), or wireless technology such as infrared, radio, or microwave signals, then the coaxial cable, fiber optic cable, twisted pair, DSL, or wireless technology are included in the definition of medium. Combinations of media are also included within the scope of computer-readable media.

In this disclosure and the following claims, the word “or” indicates an inclusive list such that, for example, the list of X, Y, or Z means X or Y or Z or XY or XZ or YZ or XYZ. Also the phrase “based on” is not used to represent a closed set of conditions. For example, a step that is described as “based on condition A” may be based on both condition A and condition B. In other words, the phrase “based on” shall be construed to mean “based at least in part on.” Also, the words “a” or “an” indicate “at least one.”

What is claimed is:

1. A method for automated patient and healthcare professional, services matching, and automated matching or generation of diagnosis, treatment plan and medication recommendation, comprising:
obtaining, by an AI empowered computer network, public information for a patient or healthcare professional, and private information for the patient or healthcare professional;
providing, by the AI empowered computer network, public and private information in response to a request for information about the patient, the healthcare professional, or the institution, and regarding one or more of:
a treatment,
a medication regarding any symptom associated with the patient, or
a medical condition.

2. The method of claim 1, wherein the public and private information comprises a ranking of a plurality of healthcare professionals, diagnosis, treatments, or medications based on a likelihood of a match with the patient.

3. The method of claim 1, further comprising:
receiving user information, wherein the public and private information is based on the user information.

4. The method of claim 1, further comprising:
computing a plurality of component rating factors using a plurality of machine learning models; and
combining the plurality of component rating factors using an ensemble algorithm to generate the public and private information.

5. The method of claim 1, further comprising:
identifying a user based on the generated public and private information, wherein the private information is provided to the user in response to the verified request.

6. A method for automated patient and healthcare professional, services matching, and automated matching or generation of diagnosis, treatment plan and medication recommendation, comprising:
obtaining, by a smart contract operating on a blockchain-based distributed computer network, public information for a patient or healthcare professional, and private information for the patient or healthcare professional;
providing, by the smart contract on the blockchain-based network, the public information via a public access layer of the blockchain-based distributed computer network in response to a public request for information about the patient or the healthcare professional; and
providing, by the smart contract on the blockchain-based network, the private information via a privileged access layer of the blockchain-based distributed computer network in response to a verified request for information about the patient or healthcare professional, wherein the patient controls data access to the privileged layer; and
providing, by the smart contract on the blockchain-based network, one or more of the public information or the private information via one or more of the public access layer or the privileged access layer of the blockchain-based distributed computer network in response to a verified request for information regarding one or more of:
a treatment,
a medication regarding any symptom, or
a medical condition.

7. The method of claim 6, further comprising:
performing, by the smart contract, a private healthcare service via the privileged access layer of the blockchain-based distributed computer network, wherein the private healthcare service runs diagnosis, and treatment for patient.

8. The method of claim 6, further comprising:
receiving, by the smart contract, a first verification from a first user via the privileged access layer of the blockchain-based distributed computer network; and
receiving, by the smart contract, a second verification from a second user via the privileged access layer of the blockchain-based distributed computer network,
wherein the private healthcare service is performed based on the first verification and the second verification.

9. The method of claim 6, further comprising:
generating, by a machine learning model operating off of the blockchain-based distributed computer network, a matching prediction in response to the verified request, the matching prediction matching the patient with one or more of a healthcare professional, a diagnosis, a treatment, or a medication; and
providing a result of the matching prediction to the smart contract, wherein the private information is based on the result of the matching prediction.

10. The method of claim 6, further comprising:
restricting access to the private information via the public access layer of the blockchain-based distributed computer network.

11. The method of claim 6, further comprising:
transferring, by the smart contract, a monetary value via the privileged access layer of the blockchain-based distributed computer network in response to a verified healthcare service.

12. The method of claim 6, further comprising:
generating, by the smart contract, a treatment request for the patient or healthcare professional; and
modifying the diagnosis, or treatment in response to feedback from patient or healthcare professional.

13. The method of claim 6, further comprising:
identifying a sender of the verified request; and
determining an authorization of the sender, wherein the private information is based on the authorization.

14. A non-transitory computer-readable medium storing instructions that, when executed by one or more processors, cause the one or more processors to perform operations comprising:
receiving patient data comprising medical history, symptoms, and demographic information;
analyzing the patient data using an artificial intelligence (AI) model trained on a dataset of historical patient records and medical outcomes;
generating, based on the analysis, a ranked list of potential diagnoses for the patient;
identifying, using the AI model, a set of suitable healthcare professionals for the patient based on the potential diagnoses and healthcare professional specialties;
matching the patient with a recommended healthcare professional from the set of suitable healthcare professionals based on one or more of availability, patient location, and historical success rates for treating similar conditions;
generating a personalized treatment plan for the patient using the AI model, wherein the treatment plan is also based on the recommended healthcare professional's expertise and the patient's specific medical profile;
transmitting the potential diagnoses, recommended healthcare professional, and personalized treatment plan to a user device associated with the patient; and
updating the AI model based on feedback received regarding the accuracy of the diagnoses, effectiveness of the doctor match, and success of the treatment plan.

15. The non-transitory computer-readable medium of claim 14, wherein the operations further comprise:
generating, using the AI model, a ranked list of recommended medications based on the potential diagnoses, the patient's medical history, and known drug interactions; and
transmitting the ranked list of recommended medications to the user device associated with the patient.

16. The non-transitory computer-readable medium of claim 14, wherein matching the patient with the recommended healthcare professional further comprises:
calculating a compatibility score between the patient and each healthcare professional in the set of suitable healthcare professionals based on factors including doctor communication style, patient preferences, and historical patient satisfaction ratings; and
selecting the healthcare professional with the highest compatibility score as the recommended healthcare professional.

17. The non-transitory computer-readable medium of claim 14, wherein the operations further comprise:
generating a blockchain-based health record for the patient, wherein the blockchain-based health record includes the patient data, potential diagnoses, recommended healthcare professional, and personalized treatment plan; and
granting selective access to portions of the blockchain-based health record to authorized healthcare providers based on patient consent and predefined access rules.

18. The non-transitory computer-readable medium of claim 14, wherein the operations further comprise:
generating, using natural language processing techniques, a summary of the patient's medical history and current symptoms from unstructured clinical notes;
incorporating the generated summary into the analysis performed by the AI model to improve the accuracy of the potential diagnoses and treatment plan recommendations.

19. The non-transitory computer-readable medium of claim 14, wherein the operations further comprise:
analyzing medical imaging data associated with the patient using computer vision techniques to identify potential abnormalities or areas of concern; and
integrating the results of the medical imaging analysis with the patient data to provide a more comprehensive input for the AI model's analysis and recommendations.

20. The non-transitory computer-readable medium of claim 14, wherein the operations further comprise:
monitoring real-time patient data from wearable devices or remote monitoring systems;
continuously updating the personalized treatment plan based on the real-time patient data and feedback from the recommended healthcare professional; and
alerting the patient and healthcare professional of any significant changes in the patient's condition or recommended treatment adjustments.
